(19) United States
(12) Patent Application Publication
(10) Pub. No.: US 2008/0146801 A1
(43) Pub. Date: Jun. 19, 2008

(54) METHOD OF PROCESSING SUBSTRATE AND CHEMICAL USED IN THE SAME

(75) Inventor: Shusaku KIDO, Kagoshima (JP)

Correspondence Address: SUGHRUE MION, PLLC, 2100 PENNSYLVANIA AVENUE, N.W., SUITE 800, WASHINGTON, DC, 20037, US

(73) Assignee: NEC LCD TECHNOLOGIES, LTD., Kanagawa (JP)

(21) Appl. No.: 12/033,643

(22) Filed: Feb. 19, 2008

Related U.S. Application Data
(62) Division of application No. 10/942,917, filed on Sep. 17, 2004.

(30) Foreign Application Priority Data
Sep. 18, 2003 (JP) 2003-326553
Nov. 5, 2003 (JP) 2003-375975
Aug. 6, 2004 (JP) 2004-230633

(51) Int. Cl. C07D 213/04; C07C 215/02

(52) U.S. Cl. 546/1; 564/503

(57) ABSTRACT

A method of processing a substrate, including a step of processing an organic film pattern formed on a substrate, the step including, in sequence, a removal step of removing one of an alterated layer and a deposited layer formed on the organic film pattern, and a fusion/deformation step of fusing the organic film pattern for deformation, wherein at least a part of the removal step is carried out by applying chemical to the organic film pattern.

### BACKGROUND OF THE INVENTION

1. Field of the Invention

The invention relates to a method of processing a substrate, and chemicals used in the method.

2. Description of the Related Art

A wiring in a circuit has been conventionally formed, for instance, by forming an organic film pattern on a semiconductor wafer, a liquid crystal display (LCD) substrate and other substrates, and etching an underlying film or the substrate with the organic film pattern being used as a mask to thereby pattern the underlying film. After an underlying film has been patterned, the organic film pattern is removed.

For instance, Japanese Patent Application Publication No. 2002-334830 has suggested a method of processing a substrate, including the steps of processing an underlying film, deforming an organic film pattern, etching the underlying film with the deformed organic film pattern being used as a mask, and removing the organic film pattern.

Specifically, the suggested method includes an ashing step to be carried out prior to the step of deforming the organic film pattern for removing an alterated or deposited layer of the organic film pattern, or improving wettability of a portion of a surface of a substrate not covered with the organic film pattern. Hereinbelow, the step of deforming the organic film pattern is referred to as a fusion/deformation step, or a gas atmosphere step because the organic film pattern is deformed by exposing a substrate to gas atmosphere.

The suggested method mainly includes the ashing step and the fusion/deformation step.

In order to stably carry out those steps, the method may include steps of controlling (specifically, lowering) a temperature of a substrate to a suitable temperature, and baking the organic film pattern after the organic film pattern has been deformed.

FIG. 1 is a flow-chart showing steps to be carried out in the method.

As illustrated in FIG. 1, the conventional method includes, in sequence, the step of ashing (step S101), controlling a temperature of a substrate (step S102), exposing a substrate to gas atmosphere (step S103), and a heating a substrate to bake the organic film pattern (step S104).

As ashing, there may be carried out dry steps such as discharging plasma in oxygen or oxygen/fluorine atmosphere, applying optical energy of a light having a short wavelength such as ultra-violet ray, or applying optical energy or heat.

An alterated layer formed on a surface of an organic film pattern, to be removed by ashing, is caused by aging, thermal oxidation, thermal hardening, adhesion of a deposited layer, wet-etching using acid etchant, oxygen ashing, and dry-etching using dry-etching gas. That is, an organic film pattern is physically and chemically damaged by those causes, and alterated. A degree of alteration and a characteristic of an alterated layer depend highly on a chemical to be used in wet-etching, whether dry-etching is isotropic or anisotropic, whether deposition exists on an organic film pattern, and gas used in dry-etching. Hence, difficulty in removing an alterated layer depends also on those.

A deposited layer formed on a surface of an organic film pattern, to be removed by ashing, is caused by dry-etching. A characteristic of a deposited layer depends on whether dry-etching is isotropic or anisotropic, and gas used in dry-etching. Hence, difficulty in removing a deposited layer depends also on those.

Ashing as a dry step can be grouped into two types.

A first type of ashing is a step other than plasma-discharging steps. For instance, a first type of ashing is comprised of a step of applying optical energy of a light having a short wavelength such as ultra-violet ray, or heat to an object such as an organic film or an underlying film. The first type of ashing exerts less damage on an object, but has a low processing speed. Accordingly, the first type of ashing is used merely for changing a surface condition of an organic film pattern or an underlying film, and is hardly used for a process required to be carried out at a high rate, such as removal of an alterated layer formed on an organic film.

A second type of ashing is a plasma-discharging step. A plasma-discharging step is grouped further into two types (hereinbelow, referred to as “type one” and “type two”). A type one is an isotropic plasma-discharging step to be carried out under a high pressure with low power, and a type two is an anisotropic plasma-discharging step to be carried out under a low pressure with high power. Both of the type one and two have a process speed higher than that of the first type of ashing, that is, a step other than plasma-discharging steps, and the type two has a higher process speed than the same of the type one. Thus, since the type one and two have a high process speed, an organic film pattern can be etched in a short period of time, and a surface of an underlying film can be changed in a short period of time. In addition, the type one and two can be carried out for removal of an alterated layer formed on a surface of an organic film pattern, or a high-speed process such as dry peeling-off. However, the second type of ashing, that is, a plasma-discharging step exerts more damage to an object than the first type of ashing.

In particular, an alterated layer formed on an organic film pattern cannot be sufficiently removed by the first type of ashing. An anisotropic plasma-discharging step (type two) can sufficiently remove an initially formed alterated layer, but would exert much damage to an organic film pattern, and resultingly, an alterated layer is newly formed on the organic film pattern. Accordingly, it is meaningless to select an anisotropic plasma-discharging step (type two) for removing an alterated layer formed on a surface of an organic film pattern. Thus, an isotropic plasma-discharging step (type one) is usually selected for removing an alterated layer formed on a surface of an organic film pattern.

However, in the method suggested in the above-mentioned Publication, when an alterated layer formed on a surface of an organic film pattern is removed for uniformizing a step of causing chemical (for instance, organic solvent) to percolate into an organic film pattern for deforming the organic film pattern, it would be impossible to completely remove the alterated layer even by the anisotropic plasma-discharging step (type two) and the isotropic plasma-discharging step (type one), and it would be also impossible to prevent a small alterated layer from being formed on an organic film pattern due to damage newly exerted by the anisotropic and isotropic plasma-discharging step.

The inventor found out the problem that even such a small alterated layer newly formed due to the plasma-discharging step prevents uniformity of a step of causing chemical to percolate into an organic film pattern for deforming the organic film pattern.

That is, the method suggested in the above-mentioned Publication is accompanied with a problem that since a step of causing chemical percolating into an organic film pattern is insufficiently carried out as a result that the organic film pattern is damaged by a plasma-discharging step and that a small alterated film is newly formed on the organic film pattern, a step of etching an underlying film is insufficiently carried out.

Japanese Patent Application Publication No. 2002-534789 based on WO00/41048 (PCT/US99/28593) has suggested an apparatus for synchronizing systems for processing a substrate. Specifically, the apparatus includes a wafer cluster tool having a scheduler which synchronizes all events in a system with one another.

Japanese Patent Application Publication No. 10-247674 has suggested an apparatus for processing a substrate, including a plurality of processors each applying a series of steps to the substrate, and a carrier carrying the substrate to each of the processors. The carrier includes a carrier plate, a first rotator rotatable around a first rotation axis extending perpendicularly to the carrier plate, a first driver for rotating the first rotator, a second rotator rotatable around a second rotation axis extending perpendicularly to the first rotator, a second driver for rotating the second rotator, a substrate-holder rotatable around a third rotation axis extending perpendicularly to the second rotator, and holding the substrate, and a third driver for driving the substrate-holder.

### SUMMARY OF THE INVENTION

In view of the above-mentioned problems in the prior art, it is an object of the present invention to provide a method of processing a substrate, which is capable of preventing an organic film pattern and a substrate from being damaged.

It is also an object to provide chemical to be used in such a method.

In one aspect of the present invention, there is provided a method of processing a substrate, including a step of processing an organic film pattern formed on a substrate, the step including, in sequence, a removal step of removing one of an alterated layer and a deposited layer formed on the organic film pattern, and a fusion/deformation step of fusing the organic film pattern for deformation, wherein at least a part of the removal step is carried out by applying chemical to the organic film pattern.

Specifically, when a step of fusing and hence deforming an organic film pattern formed on a substrate (hereinafter, referred to simply as “a fusion/deformation step) is carried out, at least a part of a removal step of removing an alterated layer or a deposited layer is carried out as a pre-step by applying chemical to the organic film pattern. The present invention makes it possible to remove an alterated layer or a deposited layer without damaging a substrate and an organic film pattern, ensuring uniform fusion and deformation of the organic film pattern.

For instance, the fusion/deformation step may be carried out by causing chemical (for instance, organic solvent) to percolate into an organic film pattern formed on a surface of a substrate, and deforming (for instance, fusion/reflow) the organic film pattern. Specifically, the fusion/deformation step may be carried out by gasifying chemical (for instance, organic solvent) with N₂ bubbling, and exposing the substrate to the thus gasified chemical atmosphere.

For instance, the fusion/deformation step is carried out for enlarging an area of an organic film pattern, integrating adjacent organic film patterns with each other, planarizing an organic film pattern, and deforming an organic film pattern such that the organic film pattern acts as an electrically insulating film covering a circuit pattern formed on a substrate.

In the first aspect of the method in accordance with the present invention, the removal step may be carried out entirely by applying chemical to an organic film pattern. Specifically, the step of processing an organic film pattern formed on a substrate includes, in sequence, a removal step of removing an alterated layer or a deposited layer formed on the organic film pattern, and a fusion/deformation step of fusing the organic film pattern for deformation.

In order to stably carry out these steps, the method may additionally include steps of controlling (specifically, lowering) a temperature of a substrate to a suitable temperature, and heating the substrate to bake the organic film pattern after the organic film pattern has been deformed.

FIG. 2 is a flow-chart showing steps to be carried out in the first aspect of the method in accordance with the present invention.

As illustrated in FIG. 2, the method includes the steps, in sequence, of applying chemical to an organic film pattern (step S1), controlling a temperature of a substrate to a suitable temperature (step S2), exposing the organic film pattern to gas atmosphere (step S3) and heating the organic film pattern (step S4).

In the second aspect, the removal step is comprised of, in sequence, ashing step as a dry step, and a step of applying chemical to an organic film pattern, as a wet step. Specifically, in the second aspect of the method in accordance with the present invention, the fusion/deformation step is carried out after there has been carried out the removal step comprised of the ashing step and the step of applying chemical to an organic film pattern.

It is preferable that the ashing step is carried out for removing only a surface of an alterated or deposited layer, and the rest of the alterated or deposited layer is removed by the wet step, that is, the step applying chemical to an organic film pattern.

The second aspect of the method can shorten a period of time for ashing step in comparison with the first aspect in which the removal step is entirely carried out by ashing, and hence, a substrate and an organic film pattern are less damaged. In addition, since the ashing step is carried out prior to the step of applying chemical to an organic film pattern, it would be possible to remove an alterated or deposited layer which cannot be removed only by the step of applying chemical to an organic film pattern.

Similarly to the first aspect of the method, the second aspect of the method may include additional steps of controlling (specifically, lowering) a temperature of a substrate to a suitable temperature, and heating the substrate to bake the organic film pattern after the organic film pattern has been deformed.

FIG. 3 is a flow-chart showing steps to be carried out in the second aspect of the method in accordance with the present invention.

As illustrated in FIG. 3, the method includes the steps, in sequence, of ashing (step S7), applying chemical to an organic film pattern (step S1), controlling a temperature of a substrate to a suitable temperature (step S2), exposing the organic film pattern to gas atmosphere (step S3) and heating the organic film pattern (step S4).

The chemical used in the step of applying chemical to an organic film pattern contains at least one of alkaline chemical, acid chemical and organic solvent. For instance, the chemical may contain any one or more of alkaline chemical, acid chemical and organic solvent.

It is preferable that the organic solvent contains at least amine.

It is preferable that the chemical contains at least organic solvent and amine.

It is preferable that the alkaline chemical contains at least amine and water.

It is preferable that the chemical contains at least alkaline chemical and amine.

For instance, amine is selected from monoethyl amine, diethyl amine, triethyl amine, monoisopyl amine, diisopyl amine, triisoply amine, monobutyl amine, dibutyl amine, tributyl amine, hydroxylamine, diethylhydroxylamine, diethylhydroxylamine anhydride, pyridine, and picoline.

It is preferable that the chemical contains anticorrosive.

The third aspect of the method in accordance with the present invention is applied to an organic film pattern comprised of an organic photosensitive film. In the third aspect of the method in accordance with the present invention, the chemical is designed to have a function of developing an organic film pattern.

For instance, the chemical may be designed to contain developing agent.

As chemical having a function of developing an organic film pattern, there may be selected organic alkaline aqueous solution containing TMAH (tetramethylammonium hydroxide) in the range of 0.1 to 10.0 weight % both inclusive, or inorganic alkaline aqueous solution such as NaOH and CaOH.

In the third aspect of the method in accordance with the present invention, it is preferable that an organic film pattern is kept not exposed to light until the organic film pattern is developed.

The fourth aspect of the method in accordance with the present invention additionally includes the step of exposing an organic film pattern to light in comparison with the third aspect. The step of exposing an organic film pattern to light is carried out prior to the step of developing the organic film pattern.

In the fourth aspect of the method in accordance with the present invention, since the step of exposing an organic film pattern to light is carried out prior to the step of developing the organic film pattern, even if the organic film pattern is exposed non-uniformly to light, the organic film pattern can be exposed fully to light. This cancels non-uniformity in exposure of the organic film pattern to light, and ensures uniform development.

In the fourth aspect of the method in accordance with the present invention, it is preferable that the organic film pattern is kept not exposed to light before the organic film pattern is exposed to light.

The fifth aspect of the method in accordance with the present invention additionally includes the step of applying chemical to an organic film pattern in comparison with the third and fourth aspects. The step of applying chemical to an organic film pattern is carried out prior to the step of developing the organic film pattern.

The sixth aspect of the method in accordance with the present invention additionally includes the step of patterning an underlying film formed below an organic film pattern by etching the underlying film with the organic film pattern before or after deformed by the fusion/deformation step, in comparison with the first to fifth aspects.

In the seventh aspect of the method in accordance with the present invention, the step of patterning the underlying film, in particular, carried out before the fusion/deformation step, is carried out by wet-etching or dry-etching. The seventh aspect of the method in accordance with the present invention enables that an alterated layer formed on a surface of an organic film pattern is comprised only of an oxide film, and that the alterated film or a deposited film is less damaged and contains less deposition.

The method in accordance with the present invention may additionally include a step of heating a substrate, cooling a substrate and controlling a temperature of a substrate, to be carried out before or after each of the original steps.

For instance, the alterated layer to be removed is caused by degradation of a surface of an organic film pattern caused by being aged, thermal oxidation, thermal hardening, wet-etching to an organic film pattern with wet-etchant, ashing (for instance, plasma with O₂ gas, applying ozone and heat, applying ultra-violet ray) to an organic film pattern, or deposition caused by dry-etching an organic film pattern.

For instance, the deposited layer to be removed is caused by deposition caused by dry-etching an organic film pattern.

The removal step accomplishes one of (a) removal of an alterated or deposited layer formed on a surface of an organic film pattern, (b) selective removal of an alterated or deposited layer formed on a surface of an organic film pattern, (c) removal of an alterated layer formed on a surface of an organic film pattern, and exposure of a non-alterated portion of an organic film pattern, and (d) removal of a deposited layer formed on a surface of an organic film pattern, and exposure of an organic film pattern.

A degree of alteration and a characteristic of an alterated layer depend highly on a chemical to be used in wet-etching, whether dry-etching is isotropic or anisotropic, whether deposition exists on an organic film pattern, and gas used in dry-etching. Hence, difficulty in removing an alterated layer depends also on those. Thus, the removal step in the first to seventh aspects of the present invention is carried out in accordance with a degree of alteration and a characteristic of an alterated layer.

The method in accordance with the present invention may further include the step of heating an organic film pattern. The step of heating an organic film pattern is carried out for removing moisture, acid solution and/or alkaline solution having percolated into the organic film pattern, or for recovering adhesion between an organic film pattern and an underlying film when an adhesive force between them is reduced. For instance, an organic film pattern is heated at 50 to 150 degrees centigrade for 60 to 300 seconds.

The advantages obtained by the aforementioned present invention will be described hereinbelow.

The method in accordance with the present invention includes, in sequence, a removal step of removing one of an alterated layer and a deposited layer formed on the organic film pattern, and a fusion/deformation step of fusing the organic film pattern for deformation, wherein at least a part of the removal step is carried out by applying chemical to the organic film pattern. The method makes it no longer necessary to carry out an ashing step in the removal step, or makes it possible to shorten a period of time for an ashing step. Thus, an organic film pattern or a substrate is less damaged.

Thus, the fusion/deformation step in which an organic film pattern is enlarged with respect to an area thereof, adjacent organic film patterns are integrated with each other, an organic film pattern is planarized, or an organic film pattern is deformed such that the organic film pattern covers therewith a circuit pattern formed on a substrate, as an electrically insulating film, can be uniformly carried out.

An ashing step has been conventionally carried out prior to the fusion/deformation step for removing an alterated layer formed on an organic film pattern. However, it was not possible to completely remove an alterated layer by the ashing step, and furthermore, an organic film pattern was damaged by the ashing step with the result of formation of another alterated layer.

In contrast, in accordance with the present invention, since at least a part of the removal step is carried out by applying chemical to an organic film pattern, it would be possible to minimize damage to be exerted on a surface of an organic film pattern and a substrate. As a result, it would be possible to uniformly carry out a fusion/deformation step.

When an organic film pattern initially formed on a substrate has two or more portions having different thicknesses from one another, and when a portion having a small thickness is further thinned or removed, it is preferable to keep the substrate not exposed to a light until the organic film pattern is developed.

When a portion having a small thickness is thinned or removed in an organic film pattern having two or more portions having different thicknesses from one another, there has been conventionally carried out dry-etching using oxygen gas, or ashing (for instance, anisotropic ashing). The method in accordance with the present invention makes it possible to reduce damage to be exerted on an organic film pattern and a substrate, by carrying out a wet step, specifically, a step of applying chemical to an organic film pattern or developing an organic film pattern, and further, to accomplish carrying out a highly selective step such as further thinning a portion having a small thickness, by virtue of a difference in a developing rate caused by whether an organic film pattern is photosensitive or not.

The above and other objects and advantageous features of the present invention will be made apparent from the following description made with reference to the accompanying drawings, in which like reference characters designate the same or similar parts throughout the drawings.

### BRIEF DESCRIPTION OF THE DRAWINGS

FIG. 1 is a flow-chart showing steps to be carried out in a conventional method of processing a substrate.

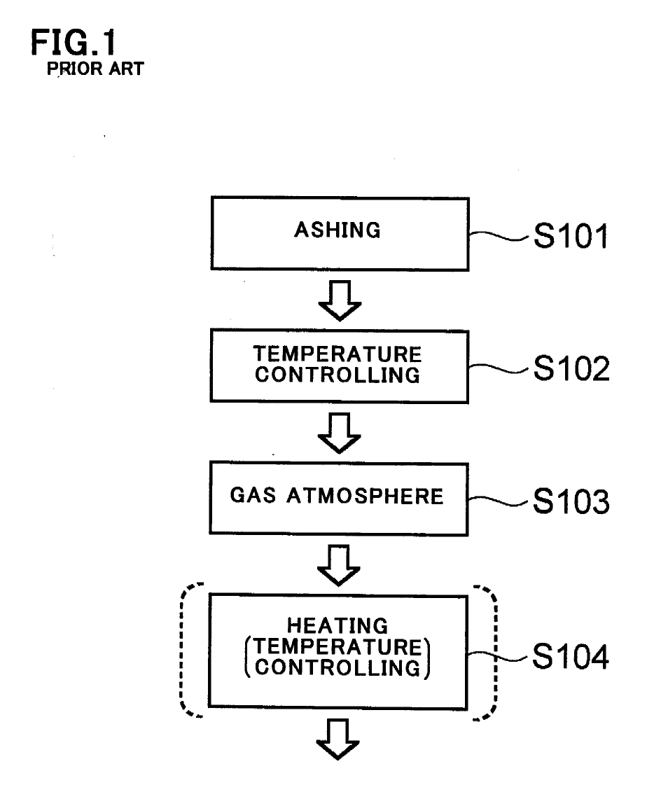

FIG. 2 is a flow-chart showing steps to be carried out in the method of processing a substrate, in accordance with the first embodiment of the present invention.

FIG. 3 is a flow-chart showing steps to be carried out in the method of processing a substrate, in accordance with the second embodiment of the present invention.

FIG. 4 is a planar view of an example of an apparatus for processing a substrate.

FIG. 5 is a planar view of another example of an apparatus for processing a substrate.

FIG. 6 is a schematic showing candidates of a process unit to be equipped in an apparatus for processing a substrate.

FIG. 7 is a cross-sectional view of an example of a unit for applying chemical to an organic film pattern.

FIG. 8 is a cross-sectional view of an example of a unit for applying gas atmosphere to an organic film pattern.

FIG. 9 is a cross-sectional view of another example of a unit for applying gas atmosphere to an organic film pattern.

FIG. 10 is a flow-chart showing steps to be carried out in the method in accordance with the third and fourth embodiments.

FIG. 11 is a flow-chart showing steps to be carried out in the method in accordance with the fifth embodiment.

FIG. 12 illustrates a degree of alteration of an alterated layer in dependence on causes by which the alterated layer is formed.

FIG. 13 is a graph showing relation between a concentration of amine in chemical and a removal rate.

FIG. 14 illustrates variation of an alterated layer to which only an ashing step is applied.

FIG. 15 illustrates variation of an alterated layer to which only a step of applying chemical is applied.

FIG. 16 illustrates variation of an alterated layer to which an ashing step and a step of applying chemical are applied in this order.

FIG. 17 illustrates a difference with respect to how an organic film pattern is processed in a fusion/deformation step.

### DESCRIPTION OF THE PREFERRED EMBODIMENTS

Preferred embodiments in accordance with the present invention will be explained hereinbelow with reference to drawings.

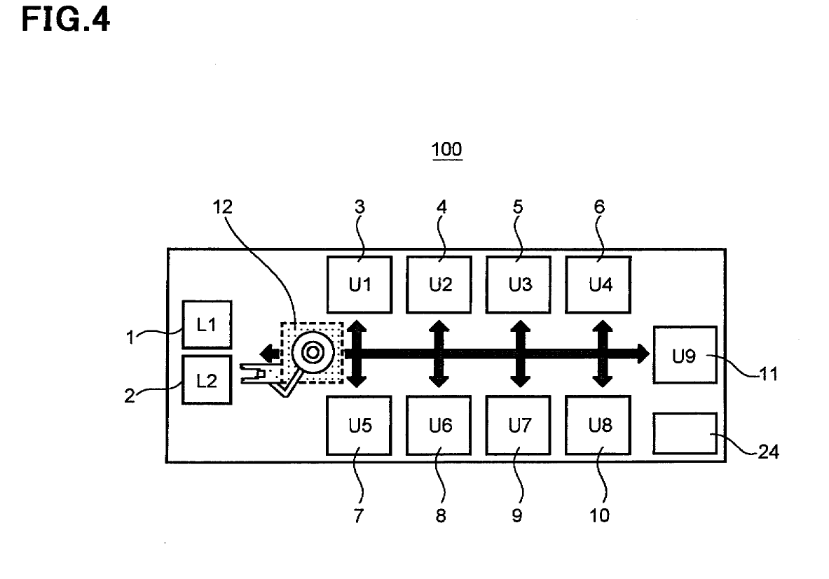

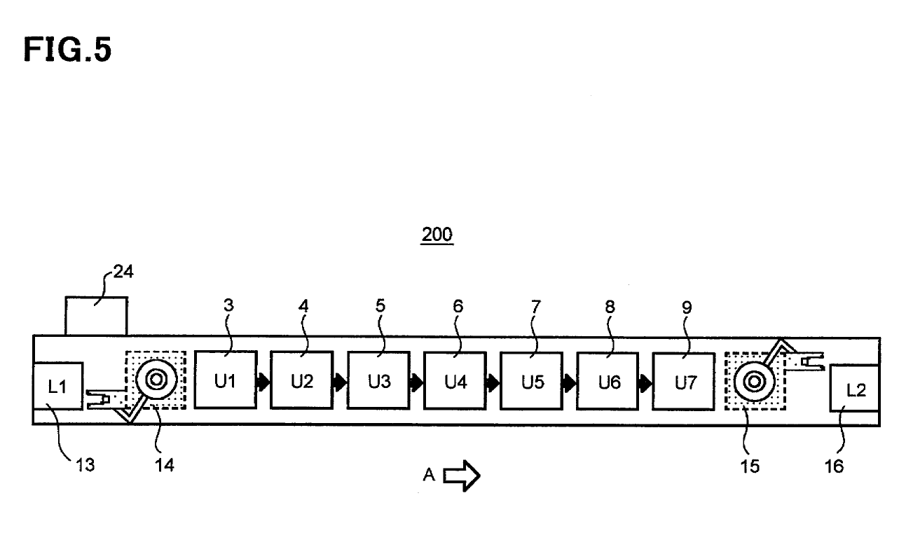

The method in accordance with the present invention is carried out in an apparatus 100 for processing a substrate, illustrated in FIG. 4 or an apparatus 200 for processing a substrate, illustrated in FIG. 5, for instance.

The apparatuses 100 and 200 are designed to be able to selectively have later-mentioned process units to apply various processes to a substrate.

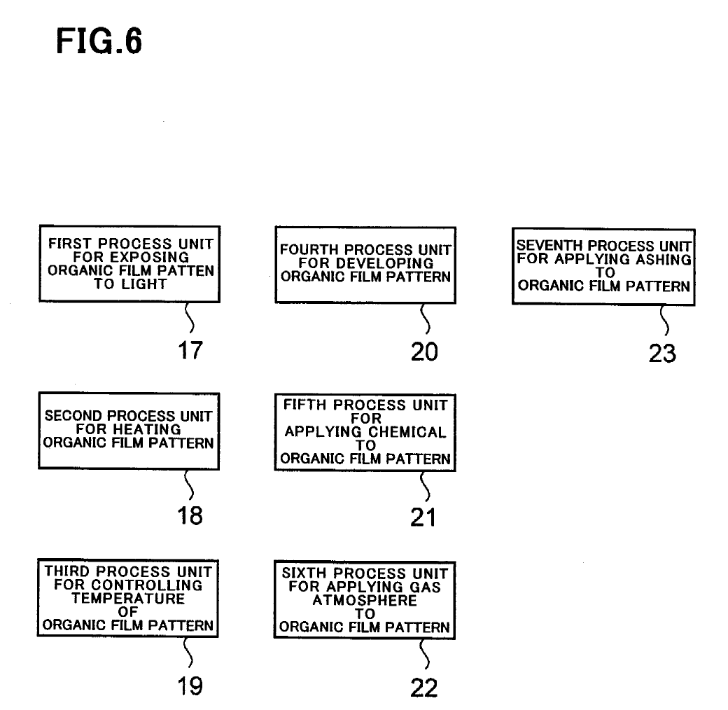

For instance, as illustrated in FIG. 6, the apparatuses 100 and 200 may include seven process units, specifically, a first process unit 17 for exposing an organic film pattern to a light, a second process unit 18 for heating an organic film pattern, a third process unit 19 for controlling a temperature of an organic film pattern, a fourth process unit 20 for developing an organic film pattern, a fifth process unit 21 for applying chemical to an organic film pattern, a sixth process unit 22 for applying gas atmosphere to an organic film pattern, and a seventh process unit 23 for applying ashing to an organic film pattern.

In the first process unit 17 for exposing an organic film pattern to a light, an organic film pattern formed on a substrate is exposed to a light. An organic film pattern covering at least a portion of a substrate therewith is exposed to a light. For instance, an organic film pattern entirely covering a substrate therewith or covering a substrate therewith in an area equal to or greater than 1/10 of a total area of the substrate is exposed to a light. In the first process unit 17, an organic film pattern may be entirely exposed to a light at a time, or a spot light may be scanned to an organic film pattern in a predetermined area. For instance, an organic film pattern is exposed to ultra-violet rays, fluorescence light or natural light.

In the second process unit 18 for heating an organic film pattern, a substrate or an organic film pattern is heated or baked in the range of 80 to 180 degrees centigrade or 100 to 150 degrees centigrade, for instance. The second process unit 18 is comprised of a stage on which a substrate is held horizontally, and a chamber in which the stage is arranged.

The third process unit 19 controls a temperature of an organic film pattern or a substrate. For instance, the third process unit 19 keeps an organic film pattern and/or a substrate in the range of 10 to 50 degrees centigrade or 10 to 80 degrees centigrade, for instance. The third process unit 19 is comprised of a stage on which a substrate is held horizontally, and a chamber in which the stage is arranged.

In the fifth process unit 21, chemical is applied to an organic film pattern or a substrate.

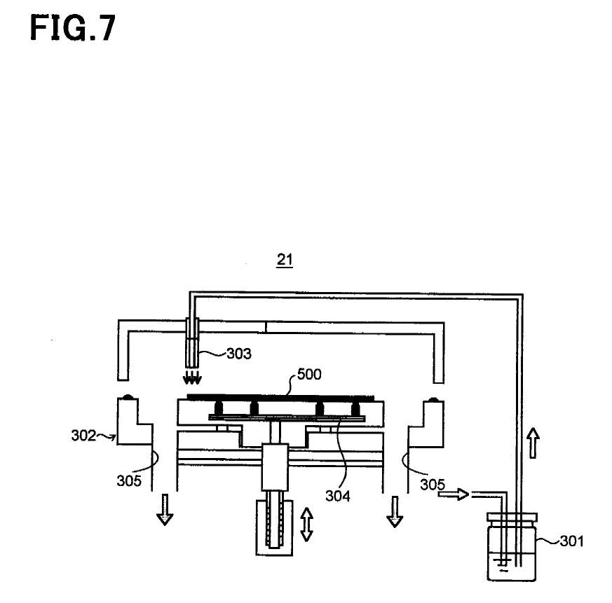

As illustrated in FIG. 7, the fifth process unit 21 is comprised of, for instance, a chemical tank 301 in which chemical is accumulated, and a chamber 302 in which a substrate 500 is arranged. The chamber 302 includes a movable nozzle 303 for supplying chemical transported from the chemical tank 301, onto the substrate 500, a stage 304 on which the substrate 500 is held almost horizontally, and an exhaust outlet 305 through which exhaust liquid and gas leave the chamber 302.

In the fifth process unit 21, chemical accumulated in the chemical tank 301 can be supplied to the substrate 500 through the movable nozzle 303 by compressing nitrogen gas into the chemical tank 301. The movable nozzle 303 is movable horizontally. The stage 304 includes a plurality of standing pins for supporting the substrate 500 at a lower surface thereof.

The fifth process unit 21 may be designed to be of a dry type in which chemical is vaporized, and the thus vaporized chemical is supplied onto the substrate 500.

For instance, chemical used in the fifth process unit 21 contains at least one of acid solution, organic solvent and alkaline solution.

In the fourth process unit 20 for developing an organic film pattern, an organic film pattern or a substrate is developed. For instance, the fourth process unit 20 may be designed to have the same structure as that of the fifth process unit 21 except that developing agent is accumulated in the chemical tank 301.

In the sixth process unit 22, there is carried out an gas atmosphere step in which various gases are applied to an organic film pattern to thereby fuse and deform the organic film pattern (fusion/deformation step).

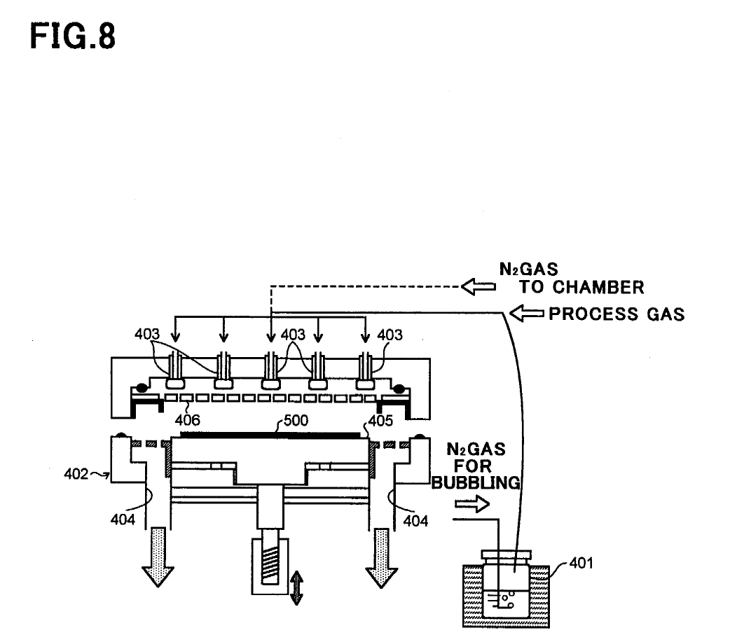

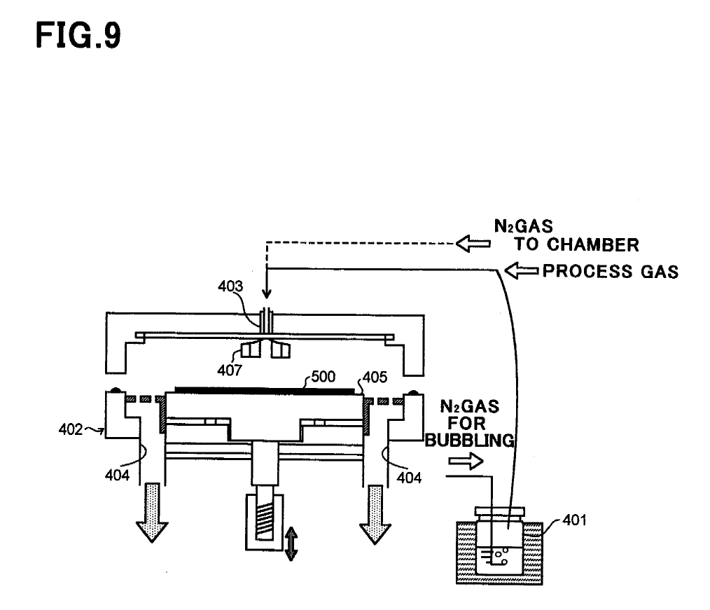

As illustrated in FIGS. 8 and 9, the sixth process unit 22 may be comprised of a container 401 in which a gas is produced by bubbling, and a chamber 402 in which a substrate 500 is arranged. The chamber 402 includes a gas inlet 403 through which a gas is introduced into the chamber 402 from the container 401, an exhaust outlet 404 through which gas is exhausted from the chamber 402, a stage 405 for almost horizontally holding the substrate 500, and a temperature controller for keeping the chamber 402 and the container 401 at a predetermined temperature. The chamber 402 may include a plurality of gas inlets 403 located at different sites from one another, and a gas distribution plate 406 having a plurality of apertures formed therethrough for dispersing and distributing gas onto the substrate 500 supported on the stage 405, as illustrated in FIG. 8. As an alternative, the chamber 402 may include a single gas inlet 403, and a distributor 407 distributing gas supplied through the gas inlet 403, by rotation, as illustrated in FIG. 9.

In the sixth process unit 22, liquid (for instance, organic solvent) accumulated in the container 401 is bubbled by introducing nitrogen gas thereinto, gas produced by bubbling the liquid is introduced into the chamber 402 through the gas inlet 403, and the substrate 500 is exposed to the gas.

In the seventh process unit 23, an organic film pattern formed on the substrate 500 is etched by plasma (oxygen plasma or oxygen/fluorine plasma), optical energy of a light having a short wavelength, such as ultra-violet ray, ozone-processing using optical energy or heat, or other steps.

As illustrated in FIG. 4, the apparatus 100 is comprised of a first cassette station 1 in which a cassette L1 in which a substrate (for instance, a LCD substrate or a semiconductor wafer) is accommodated is placed, a second cassette station 2 in which a cassette L2 similar to the cassette L1 is placed, process-unit arrangement areas 3 to 11 in each of which process units U1 to U9 is arranged, respectively, a robot 12 for transporting a substrate between the first and second cassette stations 1 and 2 and the process units U1 to U9, and a controller 24 for controlling the robot 12 to transport of a substrate and the process units U1 to U9 to carry out various processes.

For instance, substrates not yet processed by the apparatus 100 are accommodated in the cassette L1, and substrates having been processed by the apparatus 100 are accommodated in the cassette L2.

Any one of the seven process units illustrated in FIG. 6 is selected as each of the process units U1 to U9 to be arranged in the process-unit arrangement areas 3 to 11.

The number of process units is determined in accordance with a kind of process and a capacity of a process unit. Accordingly, no process unit may be arranged in any one or more of the process-units arrangement areas 3 to 11.

The controller 24 selects a program in accordance with a process to be carried out in each of the process units U1 to U9 and the robot 12, and executes the selected program to thereby control operation of the process units U1 to U9 and the robot 12.

Specifically, the controller 24 controls an order of transportation of a substrate carried out by the robot 12, in accordance with data about an order of processes, to thereby take a substrate out of the first and second cassette station 1 and 2 and the process units U1 to U9, and introduces a substrate into them in accordance with a predetermined order.

Furthermore, the controller 24 operation of the process units U1 to U9 in accordance with data about process conditions.

The apparatus illustrated in FIG. 4 is designed to be able to change an order of processes to be carried out by the process units.

In contrast, an order of processes to be carried out by the process units is fixed in the apparatus 200.

As illustrated in FIG. 5, the apparatus 200 is comprised of a first cassette station 13 in which a cassette L1 is placed, a second cassette station 16 in which a cassette L2 is placed, process-unit arrangement areas 3 to 9 in each of which process units U1 to U7 is arranged, respectively, a first robot 14 for transporting a substrate between the cassette L1 and the process unit U1, a second robot 15 for transporting a substrate between the process unit U7 between the cassette L2, and a controller 24 for controlling operation of the first and second robots 14 and 15 to transport of a substrate and the process units U1 to U7 to carry out various processes.

In the apparatus 200, an order of processes carried out in the process units U1 to U7 is fixed. Specifically, processes are continuously carried out from a process unit located upstream, that is, in a direction indicated with an arrow A.

Any one of the seven process units illustrated in FIG. 6 is selected as each of the process units U1 to U7 to be arranged in the process unit arrangement areas 3 to 9. The number of process units is determined in accordance with a kind of process and a capacity of a process unit. Accordingly, no process unit may be arranged in any one or more of the process-units arrangement areas 3 to 9.

The apparatuses 100 and 200 are designed to include a unit for transporting a substrate (specifically, the robot(s)), a unit for accommodating a cassette therein (specifically, the cassette stations), and process units selected among the seven process units illustrated in FIG. 6, in order to process an organic film pattern formed on a substrate.

Though the apparatuses 100 and 200 illustrated in FIGS. 4 and 5 are designed to include nine and seven process units, respectively, the number of process units to be included in the apparatuses 100 and 200 may be determined in accordance with a kind of a process, a capacity of a process unit, costs and so on.

Furthermore, though the apparatuses 100 and 200 are designed to include two cassettes L1 and L2, the number of cassettes may be determined in accordance with a required capacity, costs and so on.

The apparatuses 100 and 200 may include a process unit(s) other than the seven process units illustrated in FIG. 6. For instance, the apparatuses 100 and 200 may include a process unit for exposing a substrate to a light for making a minute pattern, a process unit for wet- or dry-etching a substrate, a process unit for coating a resist film onto a substrate, a process unit for strengthening an adhesion force between a substrate and an organic film pattern, or a process unit for washing a substrate (dry washing through ultra-violet ray or plasma, and wet washing through a washing agent).

If the apparatuses 100 and 200 include a process unit for wet- or dry-etching a substrate, it would be possible to pattern an underlying film (for instance, a surface of a substrate) with an organic film pattern being used as a mask.

The fifth process unit 21 may be used as a process unit for wet- or dry-etching a substrate, if the fifth process unit 21 includes chemical by which an underlying film can be etched, specifically, etchant containing acid or alkali.

In order to uniformize each of processes, the apparatuses 100 and 200 may include a plurality of common process units for applying common process to a substrate a plurality of times.

When the apparatuses 100 and 200 include a plurality of common process units for applying common process to a substrate a plurality of times, it is preferable that a substrate is processed in the common process units such that the substrate is directed in different directions from one another (for instance, oppositely) in the common process units. In such a case, the apparatuses 100 and 200 are preferably designed to have a function of directing a substrate differently in the process units, ensuring that a substrate is turned in different directions not manually, but automatically.

When the apparatuses 100 and 200 include a single process unit, it is preferable that a substrate is processed in the process unit a plurality of times with the substrate being directed in different directions from one another in each of the times. For instance, it is preferable that a substrate is processed in a plurality of directions opposite to each other, in which case, the apparatuses 100 and 200 are preferably designed to have a function of processing a substrate in a certain process unit with the substrate being directed in different directions from one another in each of the times.

It is also preferable that a substrate is processed in a process unit in a first direction and further in a second direction different from the first direction, in which case, the apparatuses 100 and 200 are preferably designed to have a function of doing so.

Hereinbelow are explained preferred embodiments in accordance with the present invention.

### First Embodiment

The first aspect of the method in accordance with the present invention is explained hereinbelow as the first embodiment of the present invention.

The first aspect of the method in accordance with the present invention is carried out for the following purposes:

(a) when an underlying film (for instance, a substrate) is etched with an organic film pattern (for instance, a resist film) being used as a mask, the underlying film is etched to be tapered, or etched in a minute size (an organic film pattern is enlarged with respect to an area, or a contact hole is reduced with respect to a size to thereby reduce an etching size);

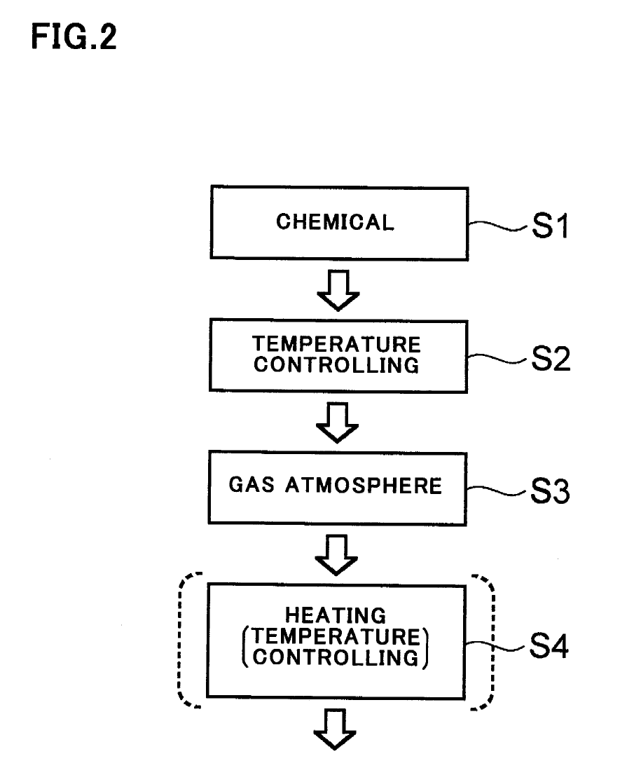

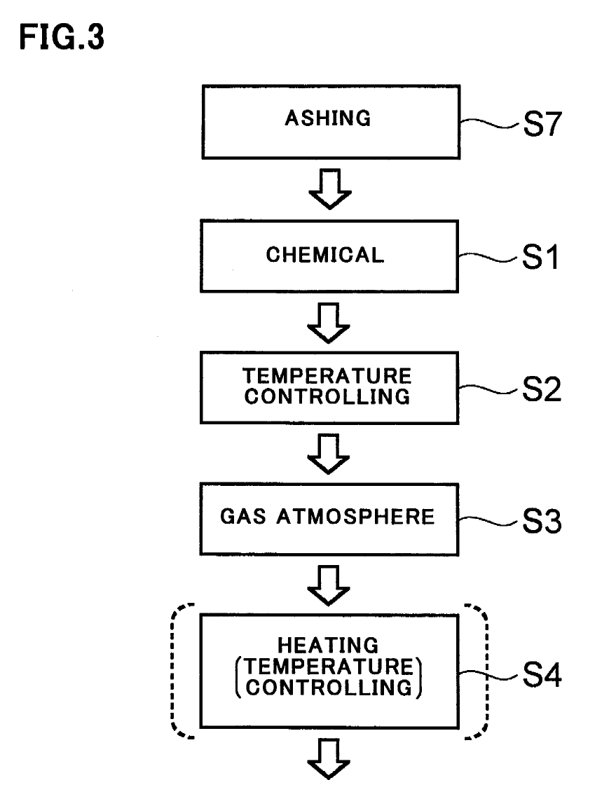

(b) when an underlying film (for instance, a substrate) is etched with an organic film pattern (for instance, a resist film) being used as a mask, the underlying film is etched into a two-layered structure, two patterns different from each other, or a combination of a separate pattern and combined patterns (for instance, see FIGS. 2 and 3 of the above-mentioned Japanese Patent Application Publication No. 2002-334830), by etching the underlying film prior to and subsequently to a fusion/deformation step; and

(c) when an organic film pattern is electrically insulating, the organic film pattern is deformed to cause the organic film pattern to act as an electrically insulating film covering a circuit pattern formed on a substrate.

The first aspect of the method in accordance with the present invention provides steps of processing an organic film pattern for accomplishing the above-mentioned purposes (a) to (c).

FIG. 2 is a flow-chart showing steps to be carried out in the method in accordance with the first embodiment of the present invention.

As illustrated in FIG. 2, the method includes the steps, in sequence, of applying chemical to an organic film pattern (step S1), controlling a temperature of a substrate or an organic film pattern to a suitable temperature (step S2), exposing an organic film pattern to gas atmosphere (step S3), and heating an organic film pattern (step S4).

The step S1 defines a removal step of removing an alterated or deposited layer, and the step S3 defines a fusion/deformation step.

The step S1 is carried out in the fifth process unit 21 for removing an alterated layer or a deposited layer formed on a surface of an organic film pattern, by applying chemical (acid solution, alkaline solution or organic solvent) to the layer.

The step S1 further improves wettability of a portion of a substrate not covered with an organic film pattern.

It is preferable in the step S1 that a period of time for carrying out the step S1 is determined or chemical is selected so as to remove only an alterated or deposited layer formed on an organic film pattern.

As a result of removal of an alterated or deposited layer, a non-alterated portion of an organic film pattern is exposed, or an organic film pattern having been covered with a deposited layer appears.

For instance, the alterated layer to be removed by the removal step (step S1) is caused by degradation of a surface of an organic film pattern caused by being aged, thermal oxidation, thermal hardening, adhesion of a deposited layer to an organic film pattern, wet-etching to an organic film pattern with acid wet-etchant, ashing (for instance, O₂ ashing) to an organic film pattern, or dry-etching through the use of dry-etching gas. That is, an organic film pattern is physically and chemically damaged by these factors, and resultingly, alterated. A degree of alteration and a characteristic of an alterated layer depend highly on a chemical to be used in wet-etching, whether dry-etching (application of plasma) is isotropic or anisotropic, whether deposition exists on an organic film pattern, and gas used in dry-etching. Hence, difficulty in removing an alterated layer depends also on those.

A deposited layer to be removed by the removal step is caused by dry-etching. A characteristic of such a deposited layer depends on whether dry-etching is isotropic or anisotropic, and gas used in dry-etching. Hence, difficulty in removing a deposited layer depends also on those.

Thus, a period of time for carrying out the step S1 and chemical to be used in the step S1 are necessary to be determined in accordance with difficulty in removing an alterated or deposited layer.

For instance, as chemical used in the step S1, there may be selected chemical containing alkaline chemical, chemical containing acid chemical, chemical containing organic solvent, chemical containing both organic solvent and amine or chemical containing alkaline chemical and amine.

For instance, the above-mentioned alkaline chemical may contain amine and water, and the above-mentioned organic solvent may contain amine.

The chemical used in the step S1 may contain anticorrosive.

For instance, amine is selected from monoethyl amine, diethyl amine, triethyl amine, monoisopyl amine, diisopyl amine, triisoply amine, monobutyl amine, dibutyl amine, tributyl amine, hydroxyl amine, diethylhydroxyl amine, diethylhydroxyl amine anhydride, pyridine, and picoline. The chemical may one or more of amine selected from them. It is preferable that the chemical contains amine in the range of 0.01 to 10 weight %.

The step S2 is carried out for keeping a temperature of a substrate or an organic film pattern at a suitable temperature prior to carrying out the step S3. For instance, a substrate or an organic film pattern is kept at 10 to 50 degrees centigrade in the step S2. In the step S2, a substrate is placed on a stage of the third process unit 19 which is kept at a predetermined temperature, and the substrate is heated until a temperature of the substrate reaches the predetermined temperature. For instance, the substrate is heated for 3 to 5 minutes.

The steps S1 and S2 provide an advantage that gas is likely to percolate into an organic film pattern in the subsequent step S3, and thus, an efficiency of the step S3 is enhanced.

In the step S3, a substrate is exposed to various gases (for instance, organic solvent) in the sixth process unit 22 for fusing and deforming an organic film pattern formed on a substrate. For instance, a substrate is exposed to gas atmosphere of organic solvent.

List 1 shows organic solvent to be preferably used in the step S3.

### [List 1]

Alcohol (R—OH)

Alkoxy alcohol

Ether (R—O—R, Ar—O—R, Ar—O—Ar)

Ester

Keton

Glycol

Alkylene glycol

Glycol ether

In List 1, R indicates an alkyl group or a substitutional alkyl group, and Ar indicates a phenyl group or an aromatic ring other than a phenyl group.

List 2 shows specific organic solvent to be preferably used in the step S3.

### [List 2]

CH₃OH, C₂H₅OH, CH₃(CH₂)XOH

Isopropyl alcohol (IPA)

Etoxy ethanol

Methoxy alcohol

Long-chain alkyl ester

Monoethanol amine (MEA)

Monoethyl amine

Diethyl amine

Triethyl amine

Monoisopropyl amine

Diisopropyl amine

Trisoproply amine

Monobutyl amine

Dibutyl amine

Tributyl amine

Hydroxyl amine

Diethylhydroxyl amine

Diethylhydroxyl amine anhydride

pyridine

picoline

acetone

Acetyl acetone

Dioxane

Ethyl Acetate

Buthyl acetate

Toluene

Methylethyl ketone (MEK)

Diethyl ketone

Dimethyl sulfoxide (DMSO)

Methylisobutyl ketone (MIBK)

Butyl carbitol

n-butylacetate (nBA)

Gammerbutyrolactone

Ethylcellosolve acetate (ECA)

Ethyl lactate

Pyruvate ethyl

2-heptanone

3-methoxybutyl acetate

Ethylene glycol

Propylene glycol

Buthylene glycol

Ethylene Glycol Monoethyl Ether

Diethylene glycol monoethyl ether

Ethylene glycol monoethyl ether acetate

Ethylene glycol monomethyl ether

Ethylene glycol monomethyl ether acetate

Ethylene glycol mono-n-buthyl ether

Polyethylene glycol

Polypropylene glycol

Polybuthylene glycol

Polyethylene glycol monoethyl ether

Polydiethylene glycol monoethyl ether

Polyethylene glycol monoethyl ether acetate

Polyethylene glycol monomethyl ether

Polyethylene glycol monomethyl ether acetate

Polyethylene glycol mono-n-buthyl ether

Methyl-3-methoxypropionate (MMP)

Propylene glycol monomethyl ether (PGME)

Propylene glycol monomethyl ether acetate (PGMEA)

Propylene glycol monopropyl ether (PGP)

Propylene glycol monoethyl ether (PGEE)

Ethyl-3-ethoxypropionate (FEP)

Dipropylene glycol monoethyl ether

Tripropylene glycol monoethyl ether

Polypropylene glycol monoethyl ether

Propylene glycol monomethyl ether propionate

3-methoxymethyl propionate

3-ethoxymethyl propionate

N-methyl-2-pyrrolidone

The step of applying gas atmosphere to a substrate through the use of gas produced from organic solvent is carried out, if an organic film pattern is fused when organic solvent percolates thereinto. For instance, an organic film pattern is soluble in water, acid and alkali, the step of applying gas atmosphere to a substrate may be carried out through the use of gas produced from aqueous solution, acid solution and alkaline solution.

In the step S4, a substrate is placed on a stage of the second process unit 18 kept at a predetermined temperature, for instance, in the range of 80 to 180 degrees centigrade, and is kept there for a predetermined period of time (for instance, 3 to 5 minutes). The step S4 allows gas to more deeply percolate into an organic film pattern, and facilitates fusion/deformation.

The apparatus 100 or 200 to be used in the first embodiment includes at least the fifth process unit 21, the third process unit 19, the sixth process unit 22, and the second process unit 18 as the process units U1 to U9 or U1 to U7.

In the apparatus 100, the fifth process unit 21, the third process unit 19, the sixth process unit 22, and the second process unit 18 may be arranged arbitrarily.

In contrast, in the apparatus 200, the fifth process unit 21, the third process unit 19, the sixth process unit 22, and the second process unit 18 are necessary to be arranged in this order in a direction indicated with an arrow A in FIG. 5. In the methods explained hereinafter, it is also necessary to arrange those process units in the order.

In accordance with the first method, the fusion/deformation step (step S3) is carried out after the step S1 has been carried out for alteration of a surface of an organic film pattern, removal of a part of a surface of an organic film pattern, or improvement of wettability of a surface of a substrate. Hence, the fusion/deformation step can be carried out controllably, uniformly and effectively, ensuring that the above-mentioned objects (a) to (c) can be accomplished.

Ashing as a dry step can be grouped into two types.

A first type of ashing is a step other than plasma-discharging steps. For instance, a first type of ashing is comprised of a step of applying optical energy of a light having a short wavelength such as ultra-violet ray, or heat to an object such as an organic film or an underlying film. The first type of ashing exerts less damage on an object, but has a low processing speed. Accordingly, the first type of ashing is used merely for changing a surface condition of an organic film pattern or an underlying film, and is hardly used for a process required to be carried out at a high rate, such as removal of an alterated layer formed on an organic film.

A second type of ashing is a plasma-discharging step. A plasma-discharging step is grouped further into types one and two. A type one is an isotropic plasma-discharging step to be carried out under a high pressure with low power, and a type two is an anisotropic plasma-discharging step to be carried out under a low pressure with high power. Both of the type one and two have a process speed higher than that of the first type of ashing, that is, a step other than plasma-discharging steps, and the type two has a higher process speed than the same of the type one. Thus, since the type one and two have a high process speed, an organic film pattern can be etched in a short period of time, and a surface of an underlying film can be changed in a short period of time. In addition, the type one and two can be carried out for removal of an alterated layer formed on a surface of an organic film pattern, or a high-speed process such as dry peeling-off. However, the second type of ashing, that is, a plasma-discharging step exerts more damage to an object than the first type of ashing.

In particular, an alterated layer formed on an organic film pattern cannot be sufficiently removed by the first type of ashing. An anisotropic plasma-discharging step (type two) can sufficiently remove an initially formed alterated layer, but would exert much damage to an organic film pattern, and resultingly, an alterated layer is newly formed on the organic film pattern. Accordingly, it is meaningless to select an anisotropic plasma-discharging step (type two) for removing an alterated layer formed on a surface of an organic film pattern. Thus, an isotropic plasma-discharging step (type one) is usually selected for removing an alterated layer formed on a surface of an organic film pattern.

However, in the method suggested in the above-mentioned Publication, when an alterated layer formed on a surface of an organic film pattern is removed for uniformizing a step of causing chemical (for instance, organic solvent) to percolate into an organic film pattern for deforming the organic film pattern, it would be impossible to completely remove the alterated layer even by the anisotropic plasma-discharging step (type two) and the isotropic plasma-discharging step (type one), and it would be also impossible to prevent a small alterated layer from being formed on an organic film pattern due to damage newly exerted by the anisotropic and isotropic plasma-discharging step.

The inventor found out the problem that even such a small alterated layer newly formed due to the plasma-discharging step prevents uniformity of a step of causing chemical to percolate into an organic film pattern for deforming the organic film pattern.

That is, the method suggested in the above-mentioned Publication is accompanied with a problem that since a step of causing chemical percolating into an organic film pattern is insufficiently carried out as a result that the organic film pattern is damaged by a plasma-discharging step and that a small alterated film is newly formed on the organic film pattern, a step of etching an underlying film is insufficiently carried out.

In accordance with the present invention, removal of an alterated or deposited layer formed at a surface of an organic film pattern, which was carried out by ashing in a conventional method, is carried out by a wet step, specifically, a step of applying chemical to an organic film pattern. Hence, it is possible to prevent an organic film pattern or a substrate from being damaged.

The step S4 of heating an organic film pattern may be omitted, in which case, it is unnecessary for the apparatus 100 or 200 to have the second process unit 18. If a temperature at which an organic film pattern is heated in the second process unit 18 can be accomplished also in the third process unit 19, the step S4 may be carried out in the third process unit 19. In FIGS. 2 to 4, a step sandwiched between parentheses may be omitted, similarly to the step S4. In addition, a process unit associated with a step sandwiched between parentheses may be also omitted.

It is preferable that a substrate is cooled down to a room temperature after the step S4 has been carried out.

Even if a common step is carried out N times (N is an integer equal to or greater than two), the apparatus 100 is not necessary to include common process units for carrying out the step, but the apparatus 200 is necessary to include N common process units for carrying out the step. For instance, if the step S4 has to be carried out twice in the apparatus 200, the apparatus 200 has to include two sixth process units 22. The same is applied to the methods explained hereinbelow.

### Second Embodiment

The second aspect of the method in accordance with the present invention is explained hereinbelow as the second embodiment of the present invention.

The second aspect of the method in accordance with the present invention is carried out for the above-mentioned purposes (a) to (c), similarly to the first embodiment. In other words, the method in accordance with the second embodiment relates to steps of processing an organic film pattern for the purposes (a) to (c).

FIG. 3 is a flow-chart showing steps to be carried out in the method in accordance with the second embodiment of the present invention.

As illustrated in FIG. 3, the method includes the steps, in sequence, of ashing an organic film pattern (step S7), applying chemical to an organic film pattern (step S1), controlling a temperature of a substrate or an organic film pattern to a suitable temperature (step S2), exposing an organic film pattern to gas atmosphere (step S3), and heating an organic film pattern (step S4).

In the second embodiment, the removal step is comprised of the ashing step (step S7) and the step of applying chemical to an organic film pattern (step S1).

The method in accordance with the second embodiment additionally includes the ashing step (step S7) which is carried out prior to the step S1. The ashing step is carried out in the seventh process unit 23.

In the ashing step, an organic film pattern is etched by plasma, optical energy of a light having a short wavelength such as ultra-violet ray, or ozone through the use of such optical energy and heat.

In the first embodiment, removal of an alterated or deposited layer formed at a surface of an organic film pattern is carried out entirely by a wet step, that is, a step of applying chemical to an organic film pattern. Unlike the first embodiment, the second embodiment includes an ashing step, a dry step, by which an alterated layer, particularly a surface of an alterated layer, is removed.

The step S1, a wet step, is carried out subsequently to the ashing step S7, a dry step, for removing an alterated layer which was not removed even by the ashing step. That is, an alterated layer formed at a surface of an organic film pattern is completely removed by a combination of the steps S1 and S7.

The steps S2, S3 and S4 are carried out in the same way as the first embodiment.

In the second embodiment, an alterated or deposited layer formed at a surface of an organic film pattern is removed by carrying out the ashing step (step S7) and the step of applying chemical to an organic film pattern (step S1), in this order. In the ashing step, only a surface of an alterated or deposited layer is removed. Hence, in comparison with a conventional ashing step, it would be possible to shorten a period of time for carrying out an ashing step, and significantly reduce damage to be exerted on an organic film pattern by an ashing step.

Even if an alterated or deposited layer cannot be removed only by the step S1, it would be possible to completely remove the layer by carrying out the ashing step S7 prior to the step S1.

As chemical to be used in the step S1 in the second embodiment, there may be selected chemical which percolate into an organic film pattern to a less degree than chemical used in the step S1 in the first embodiment, or chemical which shortens a period of time for carrying out the step S1 in comparison with the step S1 in the first embodiment.

### Third Embodiment

The third aspect of the method in accordance with the present invention is explained hereinbelow as the third embodiment of the present invention.

The method in accordance with the third embodiment is applied to an organic film pattern comprised of an organic photosensitive film. The third embodiment is different from the first and second embodiments only in that chemical used in the third embodiment is selected as chemical having a function of developing an organic film pattern.

As such chemical, there may be selected alkaline aqueous solution containing TMAH (tetramethylammonium hydroxide) in the range of 0.1 to 10.0 weight %, or inorganic alkaline aqueous solution such as NaOH or CaOH.

In the third embodiment, it is preferable that a substrate is kept not exposed to a light during initial exposure to a light for forming an organic film pattern, to development of the organic film pattern. By doing so, it would be possible to uniformize effect of development of an organic film pattern.

In order to keep a substrate not exposed to a light, all steps may be administrated for this end, or the apparatus 100 or 200 may be designed to have a function of doing so.

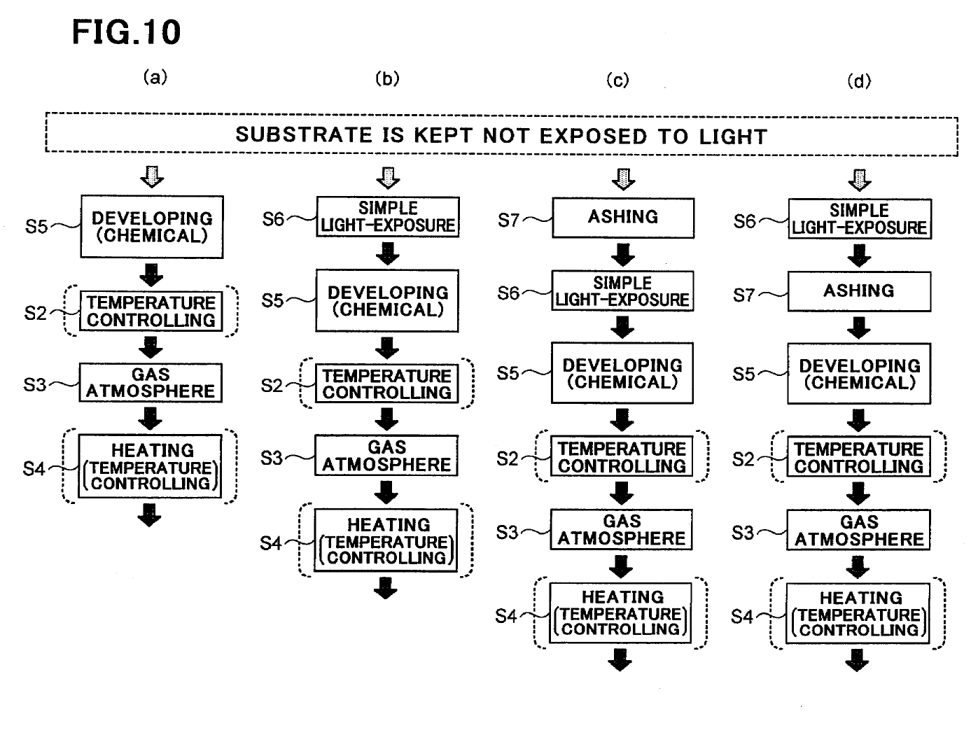

The column (a) in FIG. 10 is a flow-chart showing steps to be carried out in the method in accordance with the third embodiment.

As illustrated in the column (a) in FIG. 10, the method in accordance with the third embodiment is comprised of, in sequence, the steps of developing an organic film pattern (step S5), controlling a temperature of an organic film pattern (step S2), applying gas atmosphere to an organic film pattern (step S3), and heating an organic film pattern (step S4).

The step S5 of developing an organic film pattern defines a removal step of removing an alterated or deposited layer.

The step S5 is carried out in the fourth process unit 20. In the step S5, an organic film pattern is developed with a developing agent. The step S5 provides the same result as that of the step S1 in FIG. 2.

Accordingly, the method in accordance with the third embodiment provides the same advantages as those obtained by the method in accordance with the first embodiment.

The apparatus 100 or 200 used in the third embodiment is necessary to include the fourth process unit 20, the third process unit 19, the sixth process unit 22, and the second process unit 18 as the process units U1 to U9 or U1 to U7.

The method in accordance with the third embodiment may additionally include an ashing step to be carried out prior to the step of developing an organic film pattern (step S5), in which case, the removal step is comprised of the ashing step (step S7) and the developing step (step S5).

### Fourth Embodiment

The fourth aspect of the method in accordance with the present invention is explained hereinbelow as the fourth embodiment of the present invention.

The method in accordance with the fourth embodiment additionally includes a step of exposing an organic film pattern to a light, in comparison with the third embodiment. The step of exposing an organic film pattern to a light is carried out before a step of developing an organic film pattern.

In the step of exposing an organic film pattern to a light, an organic film pattern covering therewith a predetermined area of a substrate is exposed to a light. The step is different from a step of exposing a resist to a light for forming a minute pattern, and is referred to as “simple light-exposure step”.

The simple light-exposure step is carried out in the first process unit 17. In the first process unit 17, an organic film pattern is exposed to ultra-violet ray, fluorescent light, natural light, and other similar lights.

In the simple light-exposure step, an organic film pattern covering a part or all of a substrate therewith is exposed to a light. For instance, an organic film pattern covering 1/10 or more of a total area of a substrate therewith is exposed to a light. In the simple light-exposure step, an organic film pattern may be exposed to a light at a time, or an organic film pattern may be scanned with a spot light.

In the fourth embodiment, it is preferable that a substrate is kept not exposed to a light during initial exposure to a light for forming an organic film pattern, to development of the organic film pattern. By doing so, it would be possible to uniformize effect of development of an organic film pattern, and further uniformize exposure of a substrate to a light in the simple light-exposure step. In order to keep a substrate not exposed to a light, all steps may be administrated for this end, or the apparatus 100 or 200 may be designed to have a function of doing so.

The simple light-exposure step may be carried out in such a manner as mentioned below.

In a first case, an organic film pattern formed on a substrate kept not exposed to a light before the simple light-exposure step is carried out is exposed to a light in the simple light-exposure step.

In a second case, when a substrate is exposed to a light to some degree before the simple light-exposure step is carried out, or how degree a substrate is exposed to a light is unknown, the simple light-exposure step is carried out for entirely exposing a substrate to a light for uniformizing exposure of a substrate to a light, or for additionally exposing a substrate to a light for precaution.

### Example 1 of Fourth Embodiment

The column (b) in FIG. 10 is a flow-chart showing steps to be carried out in Example 1 of the fourth embodiment.

As illustrated in the column (b) in FIG. 10, the method in accordance with Example 1 of the fourth embodiment is comprised of, in sequence, the simple light-exposure step (step S6), the step of developing an organic film pattern (step S5), the step of controlling a temperature of an organic film pattern (step S2), the step of applying gas atmosphere to an organic film pattern (step S3), and the step of heating an organic film pattern (step S4).

The simple light-exposure step (step S6) and the step of developing an organic film pattern (step S5) define a removal step of removing an alterated or deposited layer.

The method shown in the column (b) additionally includes the simple light-exposure step (step S6) to be carried out prior to the method shown in column (a). The step S5 is effectively carried out in the method shown in the column (b), when an organic film pattern is composed of photosensitive material.

In the simple light-exposure step (step S6), an organic film pattern covering therewith a predetermined area of a substrate is exposed to a light. The step is different from a step of exposing a resist to a light for forming a minute pattern.

The simple light-exposure step is carried out in the first process unit 17. In the first process unit 17, an organic film pattern is exposed to ultra-violet ray, fluorescent light, natural light, and other similar lights.

The apparatus 100 or 200 used in Example 1 of the fourth embodiment is necessary to include the first process unit 17, the fourth process unit 20, the third process unit 19, the sixth process unit 22, and the second process unit 18 as the process units U1 to U9 or U1 to U7.

### Example 2 of Fourth Embodiment

The column (c) in FIG. 10 is a flow-chart showing steps to be carried out in Example 2 of the fourth embodiment.

As illustrated in the column (c) in FIG. 10, the method in accordance with Example 2 of the fourth embodiment is comprised of, in sequence, an ashing step (step S7), the simple light-exposure step (step S6), the step of developing an organic film pattern (step S5), the step of controlling a temperature of an organic film pattern (step S2), the step of applying gas atmosphere to an organic film pattern (step S3), and the step of heating an organic film pattern (step S4).

The ashing step (step S7), the simple light-exposure step (step S6) and the step of developing an organic film pattern (step S5) define a removal step of removing an alterated or deposited layer.

The method shown in the column (c) additionally includes the ashing step (step S7) to be carried out prior to the method shown in column (b). The ashing step S7 is carried out in the seventh process unit 23.

In Example 1, removal of an alterated or deposited layer formed at a surface of an organic film pattern is carried out entirely in a wet step, that is, the step of developing an organic film pattern. In contrast, in Example 2, the ashing step (step S7) is carried out for removing an alterated layer, particularly, a surface of an alterated layer.

In the step S5 carried out subsequently to the ashing step S7, an alterated layer which could not be removed by the ashing step is removed.

Example 2 is identical with Example 1 except the above-mentioned matters.

In accordance with Example 2, since the ashing step (step S7) is carried out prior to the step S5, an alterated layer can be effectively removed, even if an organic film pattern is cured or altered at a surface thereof due to etching having been carried out prior to the method shown in the column (c) in FIG. 10. That is, it is preferable that the ashing step S7 is applied to an organic film pattern having a surface having cured or altered due to etching.

A period of time for carrying out the ashing step S7 in Example 2 can be shortened relative to the same in the above-mentioned Japanese Patent Publication, because Example 2 has the step S5 of developing an organic film pattern.

The apparatus 100 or 200 used in Example 2 of the fourth embodiment is necessary to include the seventh process unit 23, the first process unit 17, the fourth process unit 20, the third process unit 19, the sixth process unit 22, and the second process unit 18 as the process units U1 to U9 or U1 to U7.

### Example 3 of Fourth Embodiment

The column (d) in FIG. 10 is a flow-chart showing steps to be carried out in Example 3 of the fourth embodiment.

As illustrated in the column (d) in FIG. 10, the method in accordance with Example 3 of the fourth embodiment is comprised of, in sequence, the simple light-exposure step (step S6), the ashing step (step S7), the step of developing an organic film pattern (step S5), the step of controlling a temperature of an organic film pattern (step S2), the step of applying gas atmosphere to an organic film pattern (step S3), and the step of heating an organic film pattern (step S4).

In Example 3, an order of carrying out the steps S6 and S7 is exchanged in comparison with Example 2. Example 3 provides the same advantages as the same obtained by Example 2.

The method shown in the column (d) in FIG. 10 is more suitable than Example 2, if a photosensitive organic film pattern is cured and altered in the step S6.

The apparatus 100 or 200 used in Example 3 is identical with the apparatus 100 or 200 used in Example 2.

The fourth embodiment includes the simple light-exposure step as a standard light-exposure step because of costs, a capacity, and arrangement of process units in the apparatus 100 or 200. Instead, the fourth embodiment may include a usual light-exposure step for forming a minute pattern.

The above-mentioned first to fourth embodiments shown in FIGS. 2, 3 and 10 are carried out for the purpose of (d) planarization of an organic film pattern (see Japanese Patent Application Publication No. 2003-21827, for instance), as well as for the above-mentioned purposes (a) to (c). Herein, an organic film formed on a substrate in a predetermined area may be considered as “an organic film pattern”.

When the first to fourth embodiments are carried out for the purposes (a) and (b), it is preferable to carry out a step of etching an underlying film subsequently to each of the steps or prior to and subsequently to each of the steps. Specifically, it is preferable to carry out a step of patterning an underlying film (for instance, a substrate) formed below an organic film pattern, with the organic film pattern (organic film pattern before being deformed by a fusion/deformation step) being used as a mask, or a step of patterning an underlying film (for instance, a substrate) formed below an organic film pattern, with the organic film pattern (organic film pattern after having been deformed by a fusion/deformation step) being used as a mask.

### Fifth Embodiment

The fifth aspect of the method in accordance with the present invention is explained hereinbelow as the fifth embodiment of the present invention.

The method in accordance with the fifth embodiment additionally includes a step of applying chemical to an organic film pattern, to be carried out prior to the step of developing an organic film pattern, in comparison with the methods in accordance with the third and fourth embodiments.

In the step of applying chemical to an organic film pattern, there is used chemical other than chemical having a function of development, to be used in the step of developing an organic film pattern.

### Example 1 of Fifth Embodiment

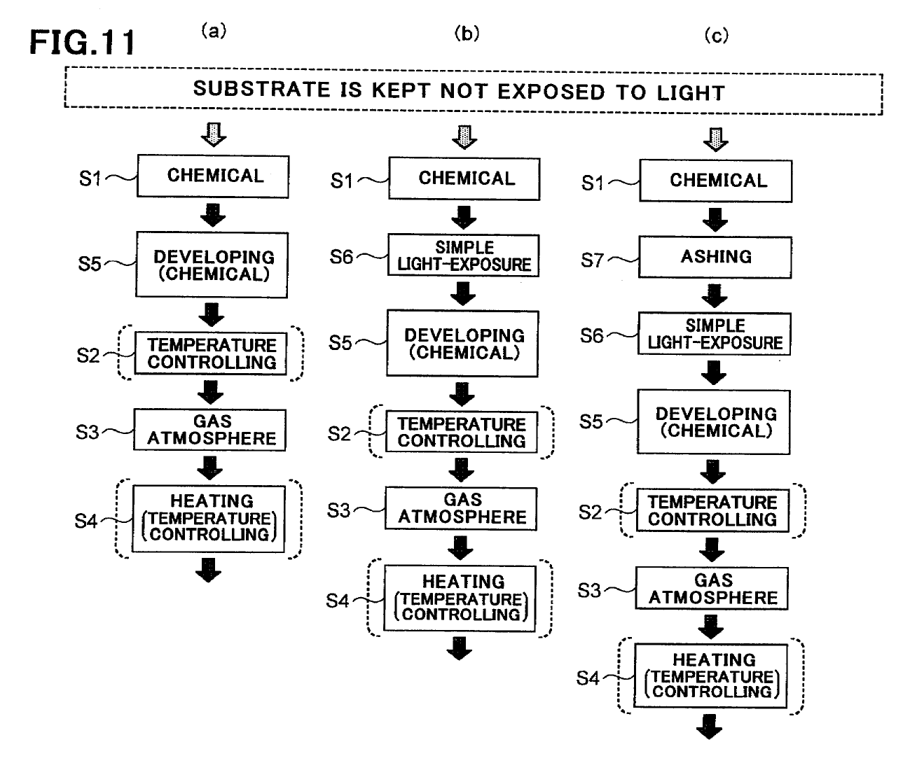

The column (a) in FIG. 11 is a flow-chart showing steps to be carried out in Example 1 of the fifth embodiment.

As illustrated in the column (a) in FIG. 11, the method in accordance with Example 1 of the fifth embodiment is comprised of, in sequence, the step of applying chemical to an organic film pattern (step S1), the step of developing an organic film pattern (step S5), the step of controlling a temperature of an organic film pattern (step S2), the step of applying gas atmosphere to an organic film pattern (step S3), and the step of heating an organic film pattern (step S4).

The step of applying chemical to an organic film pattern (step S1) and the step of developing an organic film pattern (step S5) define a removal step of removing an alterated or deposited layer.

In the step S1, there is used chemical other than chemical having a function of developing an organic film pattern.

The method in accordance with Example 1 of the fifth embodiment additionally includes the step S1 to be carried out prior to the method shown in the column (a) in FIG. 10.

That is, the method in accordance with Example 1 of the fifth embodiment improves the method shown in the column (a) in FIG. 10. The step S1 is carried out to remove a portion (in particular, a surface) of an alterated or deposited layer which could not be removed by the step of developing an organic film pattern (step S5). The step of applying chemical to an organic film pattern (step S1) is carried out in the fifth process unit 21 in the same way as the step S1 carried out in the first embodiment.

The steps S5, S2, S3 and S4 are carried out in the same way as the third embodiment.

### Example 2 of Fifth Embodiment

The column (b) in FIG. 11 is a flow-chart showing steps to be carried out in Example 2 of the fifth embodiment.

As illustrated in the column (b) in FIG. 11, the method in accordance with Example 2 of the fifth embodiment is comprised of, in sequence, the step of applying chemical to an organic film pattern (step S1), the simple light-exposure step (step S6), the step of developing an organic film pattern (step S5), the step of controlling a temperature of an organic film pattern (step S2), the step of applying gas atmosphere to an organic film pattern (step S3), and the step of heating an organic film pattern (step S4).

The step of applying chemical to an organic film pattern (step S1), the simple light-exposure step (step S6) and the step of developing an organic film pattern (step S5) define a removal step of removing an alterated or deposited layer.

In the step S1, there is used chemical other than chemical having a function of developing an organic film pattern.

The method in accordance with Example 2 of the fifth embodiment additionally includes the step S1 to be carried out prior to the method shown in the column (b) in FIG. 10.

That is, the method in accordance with Example 2 of the fifth embodiment improves the method shown in the column (a) in FIG. 10. The steps S1 and S6 are carried out to remove a portion (in particular, a surface) of an alterated or deposited layer which could not be removed by the step of developing an organic film pattern (step S5). The step of applying chemical to an organic film pattern (step S1) is carried out in the fifth process unit 21 in the same way as the step S1 carried out in the first embodiment.

The steps S5, S2, S3 and S4 are carried out in the same way as Example 1 of the fourth embodiment.

### Example 3 of Fifth Embodiment

The column (c) in FIG. 11 is a flow-chart showing steps to be carried out in Example 3 of the fifth embodiment.

As illustrated in the column (b) in FIG. 11, the method in accordance with Example 3 of the fifth embodiment is comprised of, in sequence, the step of applying chemical to an organic film pattern (step S1), the ashing step (step S7), the simple light-exposure step (step S6), the step of developing an organic film pattern (step S5), the step of controlling a temperature of an organic film pattern (step S2), the step of applying gas atmosphere to an organic film pattern (step S3), and the step of heating an organic film pattern (step S4).

The step of applying chemical to an organic film pattern (step S1), the ashing step (step S7), the simple light-exposure step (step S6) and the step of developing an organic film pattern (step S5) define a removal step of removing an alterated or deposited layer.

In the step S1, there is used chemical other than chemical having a function of developing an organic film pattern.

The method in accordance with Example 3 of the fifth embodiment additionally includes the step S1 to be carried out prior to the method shown in the column (c) in FIG. 10.

That is, the method in accordance with Example 3 of the fifth embodiment improves the method shown in the column (c) in FIG. 10. The step S1 is carried out to remove a portion (in particular, a surface) of an alterated or deposited layer which could not be removed by the step of developing an organic film pattern (step S5). The step of applying chemical to an organic film pattern (step S1) is carried out in the fifth process unit 21 in the same way as the step S1 carried out in the first embodiment.

The other steps are carried out in the same way as Example 2 of the fourth embodiment.

An order of carrying out the step S1 in the fifth embodiment is not to be limited to the orders shown in the columns (a), (b) and (c) in FIG. 11, but may be determined arbitrarily, if it is prior to the step S5. In the column (c) in FIG. 11, the ashing step S7 is carried out immediately prior to the simple light-exposure step S6. To the contrary, the ashing step S7 may be carried out immediately subsequently to the simple light-exposure step S6.

That is, for instance, there may be carried out, in sequence, the simple light-exposure step (step S6), the step of applying chemical to an organic film pattern (step S1), the step of developing an organic film pattern (step S5), the step of controlling a temperature of an organic film pattern (step S2), the step of applying gas atmosphere to an organic film pattern (step S3), and the step of heating an organic film pattern (step S4).

As an alternative, there may be carried out, in sequence, the ashing step (step S7), the simple light-exposure step (step S6), the step of applying chemical to an organic film pattern (step S1), the step of developing an organic film pattern (step S5), the step of controlling a temperature of an organic film pattern (step S2), the step of applying gas atmosphere to an organic film pattern (step S3), and the step of heating an organic film pattern (step S4).

As an alternative, there may be carried out, in sequence, the simple light-exposure step (step S6), the ashing step (step S7), the step of applying chemical to an organic film pattern (step S1), the step of developing an organic film pattern (step S5), the step of controlling a temperature of an organic film pattern (step S2), the step of applying gas atmosphere to an organic film pattern (step S3), and the step of heating an organic film pattern (step S4).

As an alternative, there may be carried out, in sequence, the ashing step (step S7), the step of applying chemical to an organic film pattern (step S1), the simple light-exposure step (step S6), the step of developing an organic film pattern (step S5), the step of controlling a temperature of an organic film pattern (step S2), the step of applying gas atmosphere to an organic film pattern (step S3), and the step of heating an organic film pattern (step S4).

In accordance with the fifth embodiment, the step of applying chemical to an organic film pattern (step S1) is carried out prior to the step of developing an organic film pattern (step S5). Hence, even if an organic film pattern is cured and altered by previous etching, a surface of the organic film pattern can be removed more effectively than the third embodiment. That is, the method in accordance with the fifth embodiment is suitable to an organic film pattern being much cured and altered.

In the above-mentioned fourth and fifth embodiments, the simple light-exposure step (step S6) may be omitted, in which case, the removal step is comprised of the steps S1 and S5 in this order or the steps S7, S1 and S5 in this order.

For instance, the simple light-exposure step (step S6) is omitted in the following two cases.

The first case is that an organic film pattern is exposed to a light in other steps or in other conditions during initial formation of an organic film pattern to processing of the organic film pattern. In the first case, even if simple light-exposure step (step S6) is omitted, it would be possible to obtain the same advantages as those provided by the fourth or fifth embodiment.

The second case is that an organic film pattern is kept not exposed to a light during initial formation of an organic film pattern to processing of the organic film pattern, and then, an alterated or deposited layer is removed by the step of applying chemical having a function of development, to the organic film pattern, and that a peripheral portion of the initial organic film pattern, exposed to a light, is removed, but a central portion of the initial organic film pattern, not exposed to a light and not alterated, should not be removed. In the second case, an alterated or deposited layer and the peripheral portion of the initial organic film pattern are simultaneously removed by the step of developing the organic film pattern and the step of applying chemical to the organic film pattern where the organic film pattern is kept not exposed to a light during initial formation of the organic film pattern to processing of the organic film pattern. As a result, the central portion of the organic film pattern which is not exposed to a light and not alterated remains as it is.

In the above-mentioned first to fifth embodiments, an organic film pattern has a uniform thickness. However, an organic film pattern may have at least two portions having different thicknesses from one another.

When an organic film pattern has at least two portions having different thicknesses from one another, it would be possible to thin a portion having a small thickness or remove a portion having a small thickness, by carrying out the step of developing an organic film pattern (step S5).

An organic film pattern having at least two portions having different thicknesses from one another can be formed by setting initial exposure of an organic film pattern to a light, at two or more levels in a plane of the organic film pattern. Specifically, there may be used two or more reticle masks allowing a light to pass therethrough in different degrees from one another.

Thereafter, a step of developing an organic film pattern (this step is different from the step S5) is carried out with the result that a portion of an organic film pattern having been exposed to a light to much or less degree has a small thickness. Thus, the organic film pattern could have portions having different thicknesses from one another.

A history of exposure of an organic film pattern to a light remains thereafter. Hence, it would be possible to further thin or remove a portion having a small thickness, by carrying out the above-mentioned development step (step S5).

As the chemical having a function of developing an organic film pattern, to be used in the step S5, if an initial organic film pattern is developed with a positive developing agent, there is used chemical having a function of positive development, and if an initial organic film pattern is developed with a negative developing agent, there is used chemical having a function of negative development.

When a portion having a small thickness among portions of an organic film pattern, having different thicknesses from one another is thinned or removed by carrying out the step of developing an organic film pattern (step S5), it is preferable that the organic film pattern is kept not to exposed to a light during initial exposure carried out for forming an organic film pattern to development of the organic film pattern.

A portion having a small thickness among portions of an organic film pattern, having different thicknesses from one another has been conventionally thinned or removed by dry-etching through the use of oxygen gas or by anisotropic ashing. In comparison with the conventional method, the methods in accordance with the above-mentioned embodiments provide the advantages that an organic film pattern and an underlying film are less damaged by the wet step, specifically, the step of applying chemical to the organic film pattern, and that a highly effective and selective step (thinning or removing a portion having a small thickness) can be carried out by virtue of a difference in a developing speed, caused by a difference as to whether an organic film pattern is photosensitive.

Hereinbelow is explained a policy as to selection of the removal step in each of the above-mentioned embodiments.

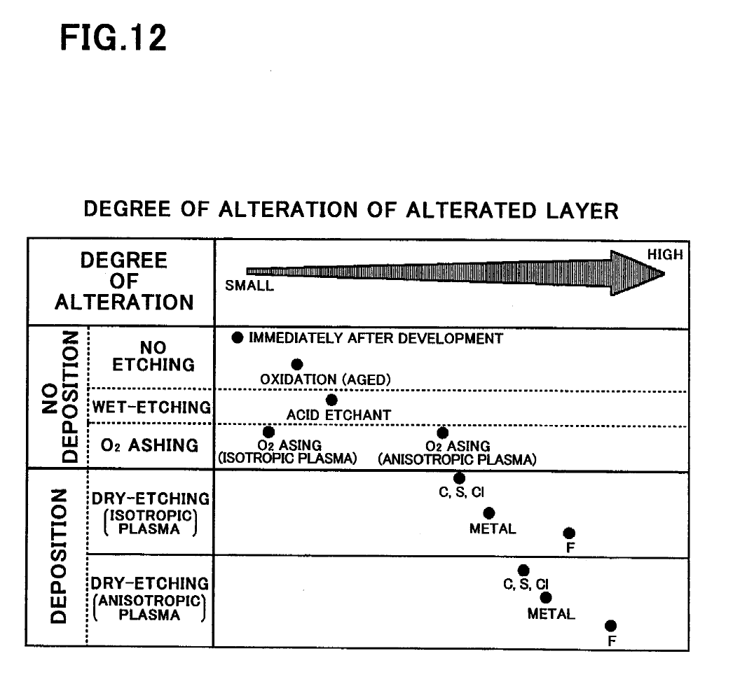

FIG. 12 illustrates a degree of alteration of an alterated layer in dependence on causes by which the alterated layer is formed. In FIG. 12, a degree of alteration is determined in accordance with difficulty in peeling off an alterated layer with a wet step.

As illustrated in FIG. 12, a degree of alteration of an alterated layer depends highly on a chemical to be used in wet-etching, whether dry-etching is isotropic or anisotropic, whether deposition exists on an organic film pattern, and gas used in dry-etching. Hence, difficulty in removing an alterated layer depends also on those.

As chemical used in the step of applying chemical to an organic film pattern, there is selected acid solution, alkaline solution or organic solvent alone or in combination.

Specifically, as the chemical is selected alkaline aqueous solution or aqueous solution containing at least one amine as organic solvent in the range of 0.05 to 10 weight %.

Herein, amine is selected from monoethyl amine, diethyl amine, triethyl amine, monoisopyl amine, diisopyl amine, triisoply amine, monobutyl amine, dibutyl amine, tributyl amine, hydroxyl amine, diethylhydroxyl amine, diethylhydroxyl amine anhydride, pyridine, or picoline.

If a degree of alteration of an alterated layer is relatively low, that is, if an alterated layer is formed due to oxidation caused by being aged, acid etchant or isotropic oxygen ashing, the selected chemical may contain amine in the range of 0.05 to 3 weight %.

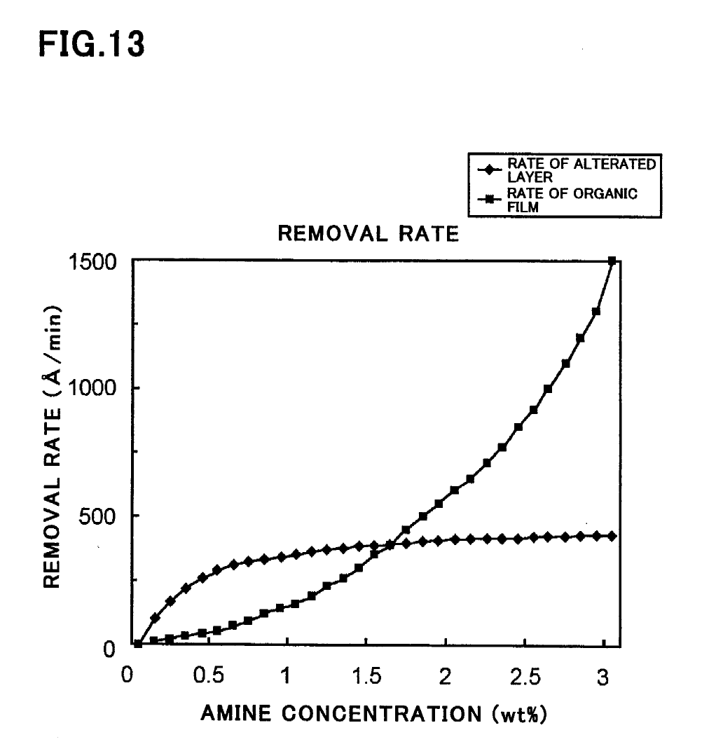

FIG. 13 is a graph showing relation between a concentration of amine in chemical and a removal rate, in association with whether an organic film pattern is alterated or not.

As illustrated in FIG. 13, it is preferable that the chemical contains amine as organic solvent in the range of 0.05 to 1.5 weight % in order to remove only an alterated layer and remain a non-alterated portion of an organic film pattern. To this end, it is preferable to select hydroxyl amine, diethylhydroxyl amine, diethylhydroxyl amine anhydride, pyridine, or picoline to be contained in the chemical. As an anticorrosive, there may be selected D-glucose (C₆H₁₂O₆), chelate or antioxidant.

By setting a suitable period of time for carrying out the step of applying chemical to an organic film pattern, as well as selecting suitable chemical, it would be possible to remove only an alterated or deposited layer, remain a non-alterated portion of an organic film pattern, or allow an organic film pattern having been covered with a deposited layer, to appear.

The step of applying chemical to an organic film pattern provides an advantage that organic solvent is likely to percolate into an organic film pattern in a fusion/deformation step to be carried out subsequently thereto.

Actually, by applying the above-mentioned chemical to an organic film pattern at a surface thereof, an alterated layer is cracked, or a part or all of an alterated layer is removed. Thus, it would be possible to avoid organic solvent from being prevented by an altered layer from penetrating an organic film pattern in a fusion/deformation step such as the step of applying gas atmosphere to an organic film pattern.

What is important is that a non-alterated portion of an organic film pattern should not be removed, but remains, and that organic solvent can readily penetrate a non-alterated portion of an organic film pattern by removing only an alterated layer or by cracking an alterated layer. It is necessary to select chemical allowing to do so.

As illustrated in FIG. 3, the columns (c) and (d) in FIG. 10, and the column (c) in FIG. 11, it is preferable that the ashing step is carried out prior to the step of applying chemical to an organic film pattern, when an alterated or deposited layer is firm or thick, or is quite difficult to remove. A combination of the ashing step and the step of applying chemical to an organic film pattern solves a problem that it is quite difficult to remove an alterated layer only by carrying out the step of applying chemical to an organic film pattern, or it takes much time to do the same.

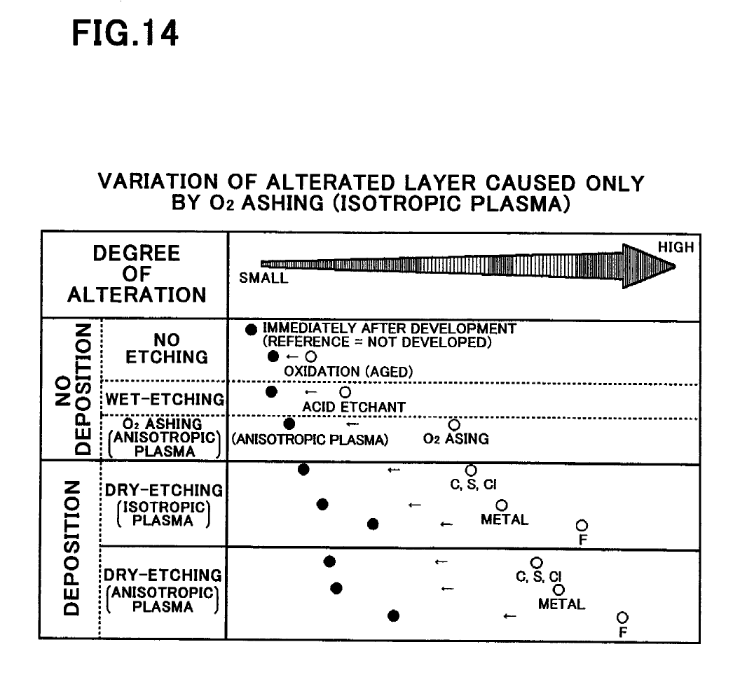

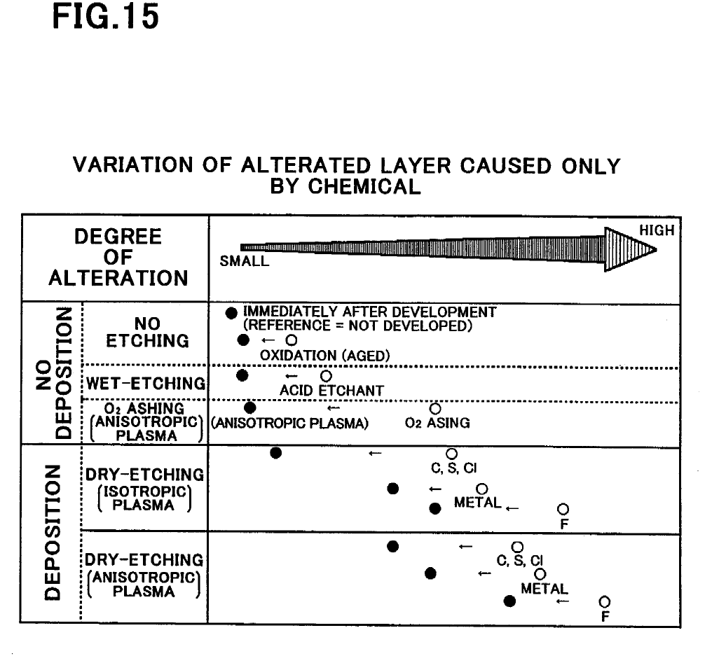

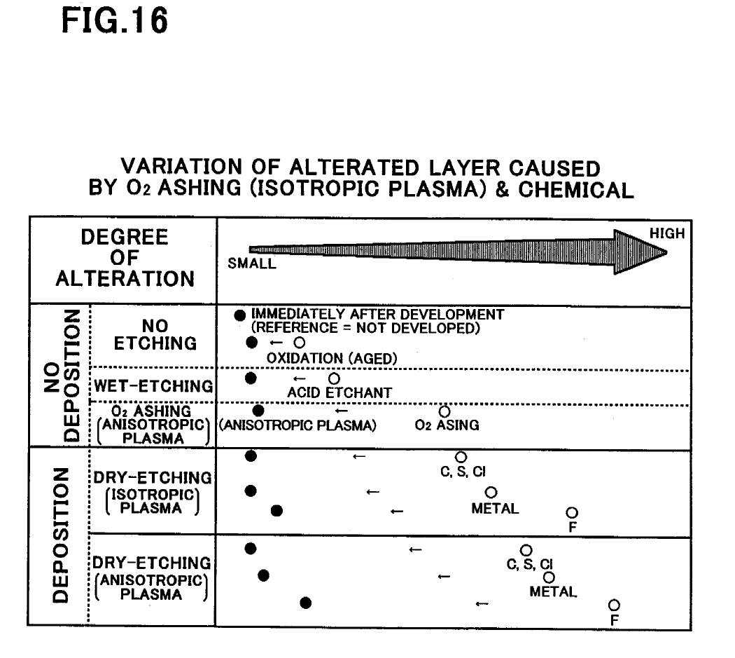

FIG. 14 illustrates variation of an alterated layer to which only an oxygen (O₂) ashing step or an isotropic plasma step is applied, FIG. 15 illustrates variation of an alterated layer to which only a step of applying chemical (aqueous solution containing hydroxyl amine at 2%) is applied, and FIG. 16 illustrates variation of an alterated layer to which both the above-mentioned ashing step and the above-mentioned step of applying chemical are applied in this order. In FIGS. 14 to 16, similarly to FIG. 12, a degree of alteration is determined in accordance with difficulty in peeling off an alterated layer with a wet step.

As illustrated in FIGS. 14 to 16, an alterated layer can be removed by carrying out any step(s). However, comparing the oxygen ashing step (isotropic plasma step) illustrated in FIG. 14 with the step of applying chemical (aqueous solution containing hydroxyl amine at 2%) to an alterated layer, a degree of removal of an alterated layer is different from each other in accordance with a thickness and characteristic of an alterated layer.

The oxygen ashing step (isotropic plasma step) is effective to removal of an alterated layer having deposition thereon, as illustrated in FIG. 14, but is likely to damage an object. Hence, if the oxygen ashing step (isotropic plasma step) is carried out to an alterated layer having no deposition thereon, an alterated layer remains without being removed to a higher degree than a degree at which an alterated layer is removed only by the step of applying chemical to an alterated layer (FIG. 15).

In contrast, the step of applying chemical (aqueous solution containing hydroxyl amine at 2%) to an alterated layer is less effective than the oxygen ashing step to removal of an alterated layer having deposition thereon, as illustrated in FIG. 15, but does not damage an object. Hence, if the step of applying chemical to an alterated layer is carried out to an alterated layer having no deposition thereon, an alterated layer remains without being removed to a higher degree than a degree at which an alterated layer is removed only by the oxygen ashing step.

Thus, in order to have the merits shown in FIGS. 14 and 15, the oxygen ashing step (isotropic plasma step) and the step of applying chemical (aqueous solution containing hydroxyl amine at 2%) to an alterated layer are carried out in this order, as illustrated in FIG. 16. It is understood that the method shown in FIG. 16 is effective to both an alterated layer having deposition thereon and an alterated layer having no deposition thereon, and can remove an alterated layer without damage remaining.

It is preferable that a layer lying below an organic film pattern is treated at a surface thereof for enhancing wettability thereof, in order to uniformize a fusion/deformation step such as a step of applying gas atmosphere to an organic film pattern. For instance, wettability of an underlying film can be enhanced by carrying out the above-mentioned ashing step, that is, the oxygen (O₂) plasma step or UV ozone treatment.

For instance, the oxygen plasma step is carried out for 120 seconds in the following conditions.

Flow rate of O₂: 300 sccm

Pressure: 100 Pa

RF power: 1000 W

The UV ozone treatment is carried out by radiating ultra-violet rays to an underlying film in ozone gas atmosphere with a temperature of a substrate being kept in the range of 100 to 200 degrees centigrade, for instance.

Wettability of an underlying film can be enhanced also by various plasma-discharge steps such as fluorine gas plasma (SF₆ gas plasma, CF₄ gas plasma, CHF₃ gas plasma, etc.) or fluorine/oxygen gas plasma (SF₆/O₂ gas plasma, CF₄/O₂ gas plasma, CHF₃/O₂ gas plasma, etc.).

These plasma steps improve wettability of a surface of an underlying film not covered with an organic film pattern. Accordingly, by carrying out these plasma steps, an organic film pattern deformed by a fusion/deformation step (for instance, the step of applying gas atmosphere to an organic film pattern) can readily reflow at a surface of an underlying film.

Pre-steps such as various plasma steps, oxygen plasma step or UV ozone step tend to damage an object in comparison with the above-mentioned step of applying chemical to an alterated layer. Hence, by removing an alterated layer by applying chemical to the alterated layer subsequently to such pre-steps as mentioned above, it would be possible to enhance wettability of an underlying film and remove an alterated layer formed at a surface of an organic film pattern, without damaging an organic film pattern. This ensures uniformly carrying out a fusion/deformation step.

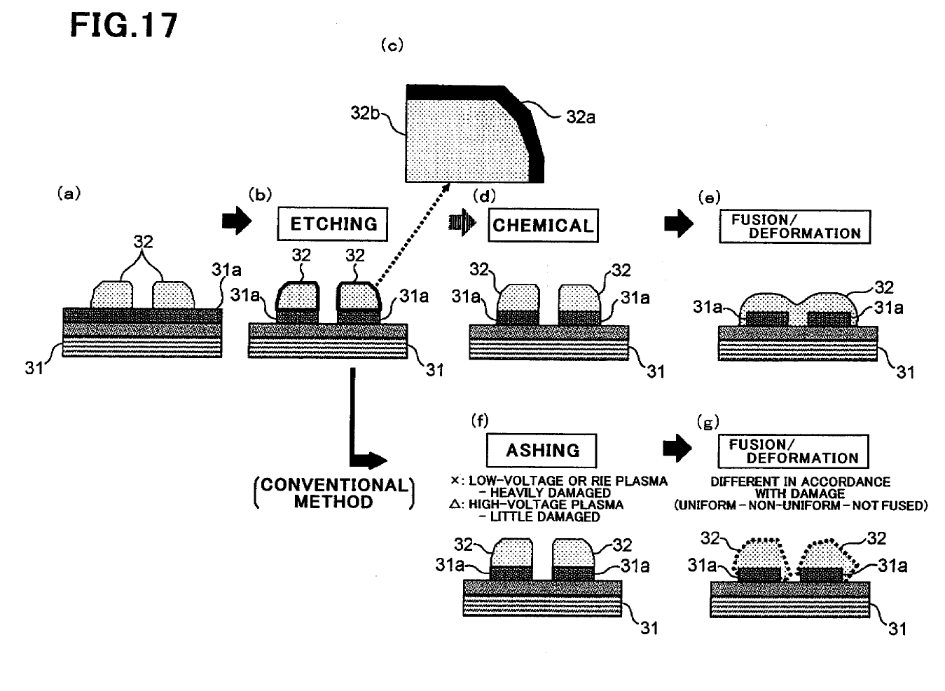

FIG. 17 illustrates the removal step in the present invention and the conventional method, to be carried out prior to a fusion/deformation step (for instance, a step of applying gas atmosphere to an organic film pattern).

FIG. 17(a) illustrates that an organic film pattern 32 is formed on a substrate 31.

FIG. 17(b) illustrates that an underlying film (for instance, an upper portion 31a of the substrate 31) is patterned by etching with the organic film pattern 32 being used as a mask.

FIG. 17(c) is an enlarged view of the organic film pattern 32 illustrated in FIG. 17(b). As illustrated in FIG. 17(c), an alterated layer 32a is formed at a surface of the organic film pattern 32, due to the etching. Hence, a non-alterated portion 32b of the organic film pattern 32 is covered with the alterated layer 32a.

FIG. 17(d) illustrates the organic film pattern 32 to which the removal step (for instance, the step of applying chemical to an organic film pattern) is applied. As illustrated in FIG. 17(d), as a result of carrying out the removal step, the alterated layer 32a is removed. The organic film pattern 32 is hardly damaged.

FIG. 17(e) illustrates the organic film pattern 32 to which a fusion/deformation step is applied subsequently to the removal step illustrated in FIG. 17(d). As illustrated in FIG. 17(e), the organic film pattern 32 is uniformly deformed by the fusion/deformation step.

FIG. 17(f) illustrates the organic film pattern 32 to which the conventional removal step (only ashing step) is applied. As illustrated in FIG. 17(f), though the alterated layer 32a is removed even by the conventional removal step, the organic film pattern 32 remains damaged.

FIG. 17(g) illustrates the organic film pattern 32 to which a fusion/deformation step is applied subsequently to the conventional removal step illustrated in FIG. 17(f). As illustrated in FIG. 17(g), the organic film pattern 32 is partially uniformly deformed by the fusion/deformation step in accordance with a degree of damage exerted on the organic film pattern 32. However, if the organic film pattern 32 is much damaged by the removal step, the organic film pattern 32 is non-uniformly deformed, or the organic film pattern 32 is not fused. Thus, it is not possible to appropriately carry out the fusion/deformation step.

The method in accordance with the present invention may include a step of heating a substrate or an organic film pattern, as a step to be first carried out. For instance, the step makes it possible to remove moisture, acid solution or alkaline solution having percolated into an organic film pattern, or to recover adhesion force between an organic film pattern and a substrate when such adhesion force is reduced. For instance, a substrate or an organic film pattern is heated for 60 to 300 seconds at 50 to 150 degrees centigrade.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

The entire disclosure of Japanese Patent Applications Nos. 2003-326553, 2003-375975 and 2004-230633 filed on Sep. 18, 2003, Nov. 5, 2003 and Aug. 6, 2004, respectively, including specification, claims, drawings and summary is incorporated herein by reference in its entirety.

What is claimed is:

1. A chemical containing amine, used in a method of processing an organic film pattern formed on a substrate, said method including, in sequence:
a removal step of removing one of an alterated layer and a deposited layer formed on said organic film pattern; and
a fusion/deformation step of fusing said organic film pattern for deformation,
wherein at least a part of said removal step is carried out by applying said chemical to said organic film pattern, and
said chemical contains said amine in the range of 0.01 to 10 weight % both inclusive.

2. The chemical as set forth in claim 1, wherein said chemical contains said amine in the range of 0.05 to 3 weight % both inclusive.

3. The chemical as set forth in claim 1, wherein said chemical contains said amine in the range of 0.05 to 1.5 weight % both inclusive.

4. The chemical as set forth in claim 1, wherein said amine is selected from a group consisting of hydroxyl amine, diethylhydroxyl amine, diethylhydroxyl amine anhydride, pyridine, and picoline.

5. A chemical containing amine, used in a method of processing an organic film pattern formed on a substrate, said method including, in sequence:
a removal step of removing an alterated layer formed on said organic film pattern to expose a non-alterated portion of said organic film pattern; and
a fusion/deformation step of fusing said organic film pattern for deformation, wherein at least a part of said removal step is carried out by applying chemical to said organic film pattern, and
said chemical contains said amine in the range of 0.01 to 10 weight % both inclusive.

6. The chemical as set forth in claim 5, wherein said chemical contains said amine in the range of 0.05 to 3 weight % both inclusive.

7. The chemical as set forth in claim 5, wherein said chemical contains said amine in the range of 0.05 to 1.5 weight % both inclusive.

8. The chemical as set forth in claim 5, wherein said amine is selected from a group consisting of hydroxyl amine, diethylhydroxyl amine, diethylhydroxyl amine anhydride, pyridine, and picoline.

9. A chemical containing amine, used in a method of processing an organic film pattern formed on a substrate, said method including, in sequence:
a removal step of removing a deposited layer formed on said organic film pattern to expose said organic film pattern; and
a fusion/deformation step of fusing said organic film pattern for deformation,
wherein at least a part of said removal step is carried out by applying chemical to said organic film pattern, and
said chemical contains said amine in the range of 0.01 to 10 weight % both inclusive.

10. The chemical as set forth in claim 9, wherein said chemical contains said amine in the range of 0.05 to 3 weight % both inclusive.

11. The chemical as set forth in claim 9, wherein said chemical contains said amine in the range of 0.05 to 1.5 weight % both inclusive.

12. The chemical as set forth in claim 9, wherein said amine is selected from a group consisting of hydroxyl amine, diethylhydroxyl amine, diethylhydroxyl amine anhydride, pyridine, and picoline.
